(19) United States
(12) Patent Application Publication
(10) Pub. No.: US 2025/0073694 A1
(43) Pub. Date: Mar. 6, 2025

(54) PLASMA-TREATED SINGLE ATOM CATALYST, PRODUCTION METHOD THEREOF AND USE OF THE CATALYST

(71) Applicant: MAX-PLANCK-GESELLSCHAFT ZUR FÖRDERUNG DER WISSENSCHAFTEN E.V., München (DE)

(72) Inventors: Beatriz ROLDAN CUENYA, Berlin (DE); Shamil SHAIKHUTDINOV, Berlin (DE); Weiming WAN, Berlin (DE)

(21) Appl. No.: 18/725,020

(22) PCT Filed: Dec. 22, 2022
(86) PCT No.: PCT/EP2022/087481

§ 371 Date: Jun. 27, 2024

(30) Foreign Application Priority Data
Dec. 30, 2021 (EP) 21218306.5

(51) Int. Cl. B01J 37/34; B01J 23/63; B01J 35/45; B01J 37/02; B01J 37/03; B01J 37/08; C01B 32/50; C07C 5/08

(52) U.S. Cl. CPC B01J 37/349; B01J 23/63; B01J 35/45; B01J 37/0217; B01J 37/0226; B01J 37/03; B01J 37/088; C01B 32/50; C07C 5/08; C07C 2523/10; C07C 2523/60; C07C 2523/62; C07C 2523/63; C07C 2523/66

(57) ABSTRACT

The present invention relates to a catalytically active material, the preparation thereof, and the use of the catalytically active material, e.g. in the catalytic oxidation of CO to CO₂ or in the catalytic hydrogenation of alkyne. The catalytically active material comprises a support5 comprising a metal oxide, and atomically dispersed noble metal on the surface of the support, wherein the metal oxide is selected from TiO₂, CeO₂, ZnO, SnO₂, Ga₂O₃, In₂O₃, ZrO₂, and Fe₂O₃, the noble metal is selected from Pt, Pd, Rh, and Au, and the catalytically active material is obtainable by a method comprising a step of non-thermal plasma treatment in the presence of O₂.

### BACKGROUND OF THE INVENTION

Catalytic selective hydrogenation of an alkyne to an alkene is an industrially important reaction. For example, the hydrogenation of acetylene to ethylene is a key process for removing acetylene from an ethylene feed for polyethylene production, since the catalyst used for the polyethylene production is poisoned by acetylene in the ethylene feed.

Catalysts based on nanoparticles containing noble metals, such as Pt and Pd, are used in the hydrogenation of alkyne. For example, the use of intermetallic Pt/Ga nanoparticles is reported in EP 2 060 323 A1.

Catalytic oxidation of CO to CO₂ is important for pollution control in the automotive industries. Noble metals, such as Pt, Pd, Rh and Au are known to be especially active in the catalytic CO oxidation reaction. Since noble metals are expensive, CO oxidation catalysts usually contain highly dispersed noble metal nanoparticles supported on metal oxides. EP 0 602 865 A1 relates, for instance, to CeO₂ as a support material for the catalyst. Compared to such nanoparticle catalysts, single atom catalysts (SACs) are generally considered to be advantageous, as they maximize the noble metal efficiency. Since catalysis is a surface reaction process, the use of smaller metal particles can save cost and/or yield better catalytic selectivity/activity.

However, as described in US 2021/0016256 A1, smaller metal particles, clusters or single atoms are not thermodynamically stable and usually sinter to form larger particles during a catalytic reaction, especially at elevated temperatures and under a reducing environment. Typical inert refractory support materials (e.g., SiO₂, Al₂O₃, etc.) have been used as high-surface-area supports, but typically do not strongly anchor small metal clusters or single metal atoms. Against this background, US 2021/0016256 A1 proposes a method to synthesize nanocomposite catalysts using the deposition of small clusters and nanoparticles of reducible oxides (CeOx, FeOx, NbOx, etc) onto inert refractory support materials selected from silica, alumina, magnesia, zirconia, cordierite, mullite, perovskite or any combination thereof. One or more atoms of Pt, Au, Pd, etc are deposited onto such “nanocomposites” via strong electrostatic adsorption (SEA) from aqueous solutions. The synthesis of the nano composite catalysts requires calcination steps in air at e.g. 400° C. or 600° C.

US 2021/0016256 A1 asserts that 0.05 wt % Pt₁/CeOx-SiO₂ or 0.05 wt % Pd₁/CeOx-SiO₂ Single-Atom Catalysts were synthesized. Increasing the loading of Pt to 2 wt. % led to the formation of Pt clusters and nanoparticles.

There is accordingly a demand of SACs with high metal loading which do not only show excellent activity and selectivity but are also stable under catalytically relevant conditions.

### Technical Problem

An object of the present invention is to provide a catalytically active material which can be effectively used for the oxidation of CO. It is a further object to provide a catalytically active material suitable for the oxidation of CO that shows a high activity and/or stability in this reaction.

According to one further aspect it is an object of the present invention to provide a catalytically active material which can be effectively used for the hydrogenation of an alkyne. It is a further object to provide a catalytically active material suitable for the hydrogenation of an alkyne that shows a high activity, high selectivity and/or stability in this reaction.

According to one further aspect, it is an object of the present invention to provide a simple and effective method for preparing such catalytically active material that can be suitably used.

### SUMMARY OF THE INVENTION

The present inventors have found that the above problem is solved by the following embodiments.
[1]A catalytically active material comprising
a support comprising a metal oxide, and
atomically dispersed noble metal on the surface of the support, wherein the metal oxide is selected from TiO₂, CeO₂, ZnO, SnO₂, Ga₂O₃, In₂O₃, ZrO₂, and Fe₂O₃, the noble metal is selected from Pt, Pd, Rh, and Au, and
the catalytically active material is obtainable by a method comprising a step of non-thermal plasma treatment in the presence of O₂.
[2] The catalytically active material according to [1], wherein
the number of the noble metal atoms normalized to the surface area of the catalytically active material is from 3.0×10¹² at/cm² to 8.0×10¹⁴ at/cm², preferably from 3.0×10¹² at/cm² to 4.0×10¹⁴ at/cm², more preferably from 6.0×10¹² at/cm² to 2.0×10¹⁴ at/cm².
[3] The catalytically active material according to [1] or [2], wherein
the relative intensity of the noble metal in oxidation state X with respect to the total intensity of the noble metal of all oxidation states in the catalytically active material is 50% or more, preferably 75% or more, as measured by X-ray photoelectron spectroscopy, wherein
X is 2+ for Pt and Pd, and X is 3+ for Rh and Au.
[4] The catalytically active material according to any one of [1] to [3], wherein an infrared absorption spectrum measured on the catalytically active material shows an absorption band within the range of 2085 cm⁻¹ to 2120 cm⁻¹ that does not change upon heating the material from 300 K to 500 K, when measured under the following condition:
   (1) exposing the catalytically active material in a reaction cell to a reaction mixture consisting of 1% CO and 5% O₂ balanced by Ar at 1 bar,
   (2) heating the catalytically active material to 500 K at a rate of 60 K/min and reacting the mixture for 5 min at 1 bar at 500 K,
   (3) cooling down the reaction mixture and the catalytically active material to 300 K,
   (4) removing the reaction mixture from the reaction cell and applying ultrahigh vacuum (UHV) conditions of 10⁻⁹ mbar or less,
   (5) recording a spectrum in UHV at 300 K without additional exposure to CO,
   (6) increasing the temperature to 500 K at a rate of 60 K/min and recording spectra at 320 K, 360 K, 400 K, 440 K, 480 K and 500 K, and
   (7) comparing the absorption maxima in the spectra recorded at the respective temperatures.
[5] The catalytically active material according to any one of [1] to [4], wherein the metal oxide and the noble metal are selected from the following material combinations:
the metal oxide is selected from TiO₂ and CeO₂ and
the noble metal from Pt and Pd, wherein
the metal oxide is preferably CeO₂ and the noble metal is preferably Pt.
[6] The catalytically active material according to any one of [1] to [5], wherein the support is in the form of particles, e.g. as powder.
[7] The catalytically active material according to [6], wherein the support consists of the metal oxide.
[8] The catalytically active material according to [6] or [7], wherein
the powder support has an average particle diameter of 10 nm to 50 nm, as measured by scanning transmission electron microscopy, and is preferably a CeO₂ powder support.
[9] The catalytically active material according to any one of [6] to [8], wherein the catalytically active material consists of the support and the noble metal.
[10] The catalytically active material according to any one of [1] to [5], wherein the support is in the form of a film.
[11] The catalytically active material according to [10], wherein
the support consists of a substrate and clusters supported on the substrate,
the substrate and the clusters comprise the same metal oxide, and
the average diameter of the clusters is 2.0 nm or less, preferably 1.5 nm or less as determined by scanning tunneling microscopy.
[12] The catalytically active material according to [11], wherein the substrate and the clusters consist of the same metal oxide.
[13] Use of the catalytically active material according to any one of [1] to [12] in the catalytic oxidation of CO to CO₂, wherein preferably the catalytically active material is used as follows:
a gas mixture comprising CO and O₂ is reacted in the presence of the catalytically active material at a pressure of 0.8 to 2.0 bar, and at reaction temperatures of 323 K to 573 K, wherein
the content of CO in the gas mixture is 5 vol % or less,
the content of O₂ in the gas mixture is 2 vol % or more and 25 vol % or less, and
the CO/O₂ molar ratio is preferably 2.0 or less.
[14] Use of the catalytically active material according to any one of [1] to [12] in catalytic hydrogenation of alkyne, wherein preferably the catalytically active material is used as follows:
a gas mixture comprising an alkyne and H₂ is reacted in the presence of the catalytically active material at a pressure of 0.8 to 2.0 bar, at a temperature of 323 K to 573 K, wherein
the content of the alkyne in the gas mixture is 10 vol % or less,
the alkyne to H₂ molar ratio in the gas mixture is 1 or less, and
the alkyne is preferably a C₂-C₅ alkyne, and more preferably a C₂-C₃ alkyne.
[15]A method for producing the catalytically active material according to any one of [6] to [9], comprising the steps of
   (1) providing a precursor for the catalytically active material comprising a support in the form of particles comprising a metal oxide and noble metal on the surface of the support,
   (2) calcining the precursor for the catalytically active material in the presence of O₂, and
   (3) carrying out a non-thermal plasma treatment of the calcined precursor for the catalytically active material in the presence of O₂.
[16] The method according to [15], wherein the step (1) is selected from the following steps (1a) and (1b):
   (1a) depositing the noble metal onto the support, preferably from an aqueous solution of a salt of the noble metal, wherein preferably the support consists of the metal oxide, and
   (1b) co-precipitating the noble metal and the metal oxide from an aqueous solution containing salts of the noble metal and the metal oxide.
[17] The method according to [15] or [16], wherein the non-thermal plasma treatment is carried out at a pressure of 20 mbar or less and at the O₂ content of 5 vol % or more.
[18]A method for producing the catalytically active material according to any one of [10] to [12] selected from Methods A and B, Method A comprising steps of
   (1) providing a metal oxide film support,
   (2) carrying out a non-thermal plasma treatment in the presence of O₂ to obtain a plasma-treated metal oxide film support, and
   (3) depositing noble metal onto the plasma-treated metal oxide film support in gas phase, preferably by physical vapor deposition of noble metal atoms; Method B comprising steps of
   (1) providing a metal oxide film support,
   (2) depositing noble metal onto the metal oxide film support from a solution containing the noble metal, and
   (3) carrying out a non-thermal plasma treatment in the presence of O₂;
wherein the non-thermal plasma treatment is preferably carried out at a pressure of 1×10⁻⁶ mbar to 1×10⁻⁵ mbar of O₂.

Where the present description refers to preferred embodiments/features including all levels of preference, combinations of these preferred embodiments/features shall also be deemed as disclosed, as long as this combination of preferred embodiments/features is technically meaningful.

Herein, the use of the term “comprising” or “including” should be understood as disclosing, as a more restricted embodiment, the term “consisting of” as well, as long as this is technically meaningful.

If preferred upper and lower limits are indicted for certain features, this should be understood as disclosing any combination of the upper and lower limits.

In the following, depending on the context, the term “catalytically active material” may also be replaced by “catalyst”.

### BRIEF DESCRIPTION OF THE DRAWINGS

FIG. 1. Schematic illustrations of the setup employed to carry out O₂-plasma treatment on samples in a film form (A) and in a particle form (B).

FIG. 2. (Left side) Low energy electron diffraction (LEED) patterns (measured at 81 eV) of the “CeO₂-plasma” film support of Reference Example 4, the “CeO₂-step” film support of Reference Example 3, the “CeO₂-red” film support of Reference Example 2, and the “CeO₂(111)” film support of Reference Example 1 (from top to bottom).

(Right side) Pt 4f X-ray photoemission spectra (XPS) obtained on the Pt/CeO₂-plasma sample of Example 1, the Pt/CeO₂-step sample of Comparative Example 3, the Pt/CeO₂-red sample of Comparative Example 2, and the Pt/CeO₂(111) sample of Comparative Example 1 (from top to bottom) before and after CO oxidation reaction (10 mbar CO, 50 mbar O₂, balanced by He to 1 bar, 523 K). The Pt 4f XPS before the reaction were measured after the samples were annealed at 523 K in ultrahigh vacuum (UHV) at a pressure of 10⁻⁹ mbar.

FIG. 3. Pt 4f, Ce 3d, and O 1s XPS spectra of the Pt/CeO₂(111) film sample according to Comparative Example 1: (i) measured at 300 K after the Pt deposition step; (ii) measured during the UHV annealing step immediately after reaching the temperature of 523 K; (iii) after the UHV annealing at 523 K for 5 min; (iv) after the CO oxidation reaction. (v) Ce 3d XPS spectra normalizing to the intensity of the peak at 882 eV. The regions of spectral changes are highlighted by grey boxes. The relative amount of Ce³⁺ is indicated adjacent to the spectra.

FIG. 4. Pt 4f, Ce 3d, and O 1s XPS spectra of the Pt/CeO₂-red film sample according to Comparative Example 2: (i) measured at 300 K after the Pt deposition step; (ii) measured during the UHV annealing step immediately after reaching the temperature of 523 K; (iii) after the UHV annealing at 523 K for 5 min; (iv) after the CO oxidation reaction. (v) Ce 3d XPS spectra normalizing to the intensity of the peak at 882 eV. The regions of spectral changes are highlighted by gray boxes. The relative amount of Ce³⁺ is indicated adjacent to the spectra.

FIG. 5. Pt 4f, Ce 3d, and O 1s XPS spectra of the Pt/CeO₂-step film sample according to Comparative Example 3: (i) measured at 300 K after the Pt deposition step; (ii) measured during the (UHV annealing step immediately after reaching the temperature of 523 K; (iii) after the UHV annealing at 523 K for 5 min; (iv) after the CO oxidation reaction. (v) Ce 3d XPS spectra normalizing to the intensity of the peak at 882 eV. The regions of spectral changes are highlighted by gray boxes. The relative amount of Ce³⁺ is indicated adjacent to the spectra.

FIG. 6. Pt 4f, Ce 3d, and O 1s XPS spectra of the Pt/CeO₂-plasma film sample according to Example 1: (i) measured at 300 K after the Pt deposition step; (ii) measured during the UHV annealing step immediately after reaching the temperature of 523 K; (iii) after the UHV annealing at 523 K for 5 min; (iv) after the CO oxidation reaction. (v) Ce 3d XPS spectra normalizing to the intensity of the peak at 882 eV. The regions of spectral changes are highlighted by gray boxes. The relative amount of Ce³⁺ is indicated adjacent to the spectra.

FIG. 7. STM images of (a) the CeO₂(111) film support according to Reference Example 1, and (b,c) the CeO₂-plasma film support according to Reference Example 4, each measured at 523 K in UHV. (d) Topography profile along the line marked in the STM image (c). (e) O 1s XPS-spectra of the CeO₂-plasma film support according to Reference Example 4, measured at normal (upper spectrum) and grazing (lower spectrum) emissions. (f) Binding energy shifts for the peroxo-O atoms at the surface of CeO₂ (open symbols) and surface 0 atoms of CeO₂ (filled symbols) referenced to the lattice 0 atoms in bulk CeO₂ (set to zero), calculated for each of the CeO₂(111), CeO₂(110) and CeO₂(100) surfaces. The latter are in abundance and dominate the experimental spectra. The shaded area highlights experimentally measured BE shifts. Insets show the top views of the topmost layer in Density Functional Theory (DFT)-optimized structures, containing ¼ monolayer of peroxide species on (111), (110), and (100) surfaces.

FIG. 8. Schematic illustrations of pristine (100), (110), (100) and (100)-40 surfaces of CeO₂ viewed from top (above) and the schematic illustrations of peroxide species formed on the same surfaces of CeO₂ by the reaction with oxygen radicals (below). The oxygen-oxygen bond length is annotated.

FIG. 9. Schematic illustrations of (111), (110), (100) and (100)-40 surfaces of CeO₂ viewed from top, with 0.5 monolayer peroxide coverage having diagonal and row-wise arrangements of the peroxide species.

FIG. 10. Infrared reflection-absorption spectra (IRAS) measured in ultrahigh vacuum on (a) the (annealed) Pt/CeO₂(111) film sample according to Comparative Example 1 and (b) the (annealed) Pt/CeO₂-plasma film sample according to Example 1 with CO adsorption.

FIG. 11. (a) Pt 4f and (b) O 1s XPS spectra of the (annealed) Pt/CeO₂-plasma film sample according to Example 1 and the (annealed) Pt/CeO₂-sputter film sample according to Comparative Example 4.

FIG. 12. (a) CO oxidation rates measured on the “Pt/CeO₂-plasma” film sample according to Example 1 and the “Pt/CeO₂(111)” film sample according to Comparative Example 1 and their comparison with “CeO₂-plasma” and “CeO₂ (111)” film samples. (b) Pt 4f XPS spectra measured on the “Pt/CeO₂-plasma” film sample according to Example 1 and the “Pt/CeO₂(111) film” sample according to Comparative Example 1 after the CO oxidation reaction.

FIG. 13. (a) CO oxidation rates measured on the plasma-treated Pt/CeO₂ powder catalyst according to Example 2 and the pristine Pt/CeO₂ powder catalyst according to Comparative Example 5 and their comparison with “CeO₂-plasma” and “CeO₂” powder samples.

Aberration corrected scanning transmission electron microscopy (STEM) images of the plasma-treated Pt/CeO₂ powder catalyst according to Example 2 before (b) and after (c) the CO oxidation reaction at 523° C. Some Pt single atoms are highlighted by circles.

FIG. 14. Pt 4f XPS spectra of (a) the plasma-treated Pt/CeO₂ powder catalysts according to Examples 2-4 and (b) the pristine Pt/CeO₂ powder catalysts according to Comparative Examples 5-7, measured after the CO oxidation reaction at 523 K for 3 h.

FIG. 15. Aberration corrected STEM images of (a) the pristine Pt/CeO₂ powder catalyst according to Comparative Example 6, (b) the plasma-treated Pt/CeO₂ powder catalyst according to Example 3, (c) the pristine Pt/CeO₂ powder catalyst according to Comparative Example 7, and (d) the plasma-treated Pt/CeO₂ powder catalyst according to Example 4, measured after the CO oxidation reaction at 523 K for 3 h. Pt single atoms are highlighted by dash-lined circles and PtOₓ clusters are highlighted by solid-lined circles.

FIG. 16. (a) Raman spectra observed for the pristine CeO₂ support according to Reference Example 5 (denoted as “CeO₂-calcined”) and the plasma-treated CeO₂ support according to Reference Example 6 (denoted as “CeO₂-plasma”). (b) Raman spectra observed for the pristine Pt/CeO₂ catalyst according to Comparative Example 5 (denoted as “Pt/CeO₂-calcined”) and the plasma-treated Pt/CeO₂ catalyst according to Example 2 (denoted as “Pt/CeO₂-plasma”). All spectra are normalized to the maximum of the principal peak at 465 cm⁻¹ and are vertically offset for clarity.

FIG. 17. Ethene production rates observed for the plasma-treated Pt/CeO₂ powder catalyst according to Example 2 (“Pt/CeO₂-pla(sma)”), the pristine Pt/CeO₂ powder catalyst according to Comparative Example 5 (“Pt/CeO₂-cal(cined)”), as well as on the Pt-free, calcined CeO₂ powder support according to Reference Example 5 (“CeO₂-cal(cined)”), and the plasma-treated CeO₂ powder support according to Reference Example 6 (“CeO₂-pla(sma)”).

FIG. 18. Pt 4f XPS spectra measured on the Pt/CeO₂-plasma film sample according to Example 1, the Pt/CeO₂(111) film sample according to Comparative Example 1, the Pt/TiO₂-plasma film sample according to Example 5, the Pt/TiO₂(110) film sample according to Comparative Example 8 after the UHV annealing at 523 K for 5 min.

FIG. 19. Schematic visualization of the restructuring of the CeO₂(111) film that results in the formation of CeO₂ nanoclusters with simultaneous reduction of the film thickness.

FIG. 20. Robustness analysis of the final input parameters used in the thermodynamic model calculations.

FIG. 21. Schematic illustrations of the energetically most favorable optimized structures of an adsorbed Pt atom on the (111), (110), (100) and (100)-40 surfaces of CeO₂ with different peroxide coverages that range from 0 (denoted “pris”) to 1.0 monolayer (denoted “1.0”), all viewed from top.

FIG. 22. Schematic illustrations of stepped (112)b, (210), (211) a, (211)b, (221), (310), (311) a and (311)b surfaces of CeO₂ and an adsorbed Pt atom for the approximation of the edge energy used in the thermodynamic model of ceria nanoparticle formation and the consideration of alternative binding sites for single-atom Pt.

FIG. 23. (a) Schematic illustration of Pt atom adsorbed on different sites on a CeO₂ nanoparticle/film model composite system (Ce₁₇₈O₃₅₆, shown in the middle). The adsorption energies (in eV; referenced to bulk Pt) are shown adjacent to the structures. (b) Schematic illustration of optimized geometries for CO adsorption on Pt single atom at the CeO₂ nanoparticle/boundary and at Pt single atom in a (100)-40 (denoted as “Pt-40”) as well as (100)-30 (denoted as “Pt-30”) and (100)-20 (denoted as “Pt-20”) sites. Pt-30 and Pt-20 sites were generated by removing one or two ligand oxygen atoms from the Pt-40. CO adsorption energies (in eV) are indicated.

FIG. 24. Schematic illustration of Pt bound at the boundary and (100)-40 on a CeO₂ nanoparticle/slab model system as well as at (100)-30 and (100)-20 sites derived from the (100)-40 site, and calculated CO adsorption energies (in eV).

FIG. 25. Schematic representation of the interaction of Pt atoms with CeO₂(111) film support and the CeO₂-plasma film support. Upon deposition on the CeO₂(111) surface, Pt forms small clusters which aggregate into larger Pt NPs at elevated temperatures (top row).

Plasma-treatment of the CeO₂(111) surface in the presence of O₂ produces peroxo species and induces surface restructuring, resulting in small ceria NPs, which act as anchoring sites either directly upon Pt adsorption or through surface migration of peroxo-stabilized Pt single atoms (bottom row).

### DETAILED DESCRIPTION OF THE INVENTION

### 1. Catalytically Active Material

The catalytically active material of the present invention comprises a support comprising a metal oxide, and atomically dispersed noble metal on the surface of the support. The support comprises a metal oxide, and the metal oxide is selected from TiO₂, CeO₂, ZnO, SnO₂, Ga₂O₃, In₂O₃, ZrO₂, and Fe₂O₃. The noble metal is selected from Pt, Pd, Rh and Au. The catalytically active material is obtained by a method comprising a step of non-thermal plasma treatment in the presence of O₂. This method comprises e.g. a step of subjecting the support comprising a metal oxide and noble metal on its surface to a non-thermal plasma treatment in the presence of O₂. In the alternative, the support comprising a metal oxide is subjected to a non-thermal plasma treatment prior to the deposition of noble metal on its surface.

As “atomically dispersed” we understand the presence of single noble metal atoms on the surface of the support which can be detected by usual analytical techniques such as extended X-ray absorption fine structure (EXAFS), infrared spectroscopy (IR), or STEM.

Analysis with STEM is further described in the Experimental Section. For the avoidance of doubt and if not stated otherwise, the term “atom” is used in the description and claims to cover both neutral (oxidation state=0) and charged (ionic, e.g. oxidized) noble metal atoms.

The form of the support is not particularly limited. The support may be, for instance, in the form of particles (e.g. powder) or a film.

The present invention is characterized in that the catalytically active material is obtained by a method comprising a step of non-thermal plasma treatment in the presence of O₂.

In one embodiment, the catalytically active material of the present invention shows a Raman band centered within the range of 815-845 cm⁻¹. The Raman spectroscopy can be carried out according to the method as described in the examples. Without being bound to theory, the Raman band centered within the rage of 815-845 cm⁻¹ is considered to be attributed to surface peroxo (O₂²⁻) species preferably formed on the surface of the metal oxide during the non-thermal plasma treatment.

In one embodiment, the catalytically active material of the present invention shows an additional O 1s XPS signal at a binding energy that is 1.2 eV to 2.0 eV, preferably 1.5 eV to 1.7 eV higher than that of the main signal of oxygen in the metal oxide in the support. The XPS analysis can be carried out according to the method as described in the examples. The additional XPS signal may be observed as a shoulder of the main signal. Without being bound to theory, the additional XPS signal is considered to be attributed to surface peroxo (O₂²⁻) species, preferably formed on the surface of the metal oxide during the non-thermal plasma treatment. For example, in the case of the CeO₂ support, the O 1s XPS signal of pristine (not plasma-treated) CeO₂ appears at 529.2 eV, while that of the surface peroxo species appears at 530.8 eV.

In one embodiment, the non-thermal plasma treatment in the presence of O₂ leads to the formation of metal oxide nanoclusters at the support surface. The average diameter of the metal oxide nanoclusters may be 2.0 nm or less, preferably 1.5 nm or less, as determined by scanning tunneling microscopy (STM); see further description below. The STM measurements can be carried out as described in the examples.

In one embodiment, the catalytically active material of the present invention shows two or more characteristics as described in the above embodiments.

According to the present invention, the support comprises a metal oxide at the surface of the support, and the metal oxide is selected from TiO₂, CeO₂, ZnO, SnO₂, Ga₂O₃, In₂O₃, ZrO₂ and Fe₂O₃. These metal oxides are frequently called reducible metal oxides. The reducible metal oxide is capable of losing an oxygen atom from its crystal structure at a relatively low energy, which results in the formation of an oxygen vacancy and the reduction in the oxidation number of the metal atom in said reducible metal oxide, e.g. from Ce⁴ to Ce³¹. The metal oxide is preferably selected from TiO₂, CeO₂, SnO₂, Ga₂O₃, In₂O₃ and Fe₂O₃, more preferably from TiO₂, CeO₂, and In₂O₃, most preferably CeO₂.

Since the support comprises the reducible metal oxide, the catalytically active material of the present invention can show excellent catalytic activity and stability and the CO oxidation reaction can be carried out at a relatively low reaction temperature. Without being bound to theory, it is believed that the metal oxide nanoclusters are formed on the support surface upon the plasma treatment in the presence of O₂, and the atomically dispersed noble metals strongly interact with the metal oxide. It is believed that this interaction allows charge transfer and the presence of an oxygen atom bridging the noble metal and the metal oxide, leading to excellent stability and excellent catalytic activity, even at a low reaction temperature.

Another benefit of using specific reducible metal oxides (instead of e.g. SiO₂) for the support is that reducible oxides, such as CeO₂ per se, may also contribute to the catalytic activity in the reaction to be catalyzed, especially if the reducible metal oxide has been plasma-treated.

In one preferred embodiment, CeO₂ clusters are created during the plasma treatment of CeO₂ support and hence, metal atoms (e.g. Pt atoms) interact with the also active CeO₂ support (when compared to e.g. SiO₂). Furthermore, the data presented in the example section show that, even in the absence of a noble metal such as Pt, the CeO₂ support may become activated by the plasma treatment. This finding can be assigned to the formation of small CeO₂ clusters with a higher surface density and possibly also the formation of peroxide surface species, e.g. on the CeO₂ clusters formed. These findings are transferable to other reducible metal oxides and noble metals as used in the present invention.

The catalytically active material according to the present invention comprises the atomically dispersed noble metals on the surface of the support, wherein the noble metal is selected from Pt, Pd, Rh and Au. Preferably, the noble metal is selected from Pt and Pd. Further preferably, the noble metal is Pt.

The combination of the metal oxide and the noble metal is not particularly limited. According to one preferred embodiment, the metal oxide is selected from TiO₂ and CeO₂, and the noble metal is selected from Pt and Pd, the combination of CeO₂ and Pt being more preferred.

The number of the noble metal atoms normalized to the surface area of the catalytically active material (surface density, Ns) is not particularly limited, and preferably at least 3.0×10¹² at/cm², more preferably at least 6.0×10¹² at/cm², further preferably at least 1.2×10¹³ at/cm². The upper limit for the Ns is 8.0×10¹⁴ at/cm², and may be for example 4.0×10¹⁴ at/cm², 2.0×10¹⁴ at/cm², 1.0×10¹⁴ at/cm², 8.0×10¹³ at/cm², 6.0×10¹³ at/cm², or 4.0×10¹³ at/cm². Any combinations of the above upper and lower limits are embodiments of the present invention. Therefore, the Ns may be, for example, 3.0×10¹² at/cm² to 8.0×10¹⁴ at/cm², 3.0×10¹² at/cm² to 4.0×10¹⁴ at/cm², 3.0×10¹² at/cm² to 2.0×10¹⁴ at/cm², 6.0×10¹² at/cm² to 8.0×10¹⁴ at/cm², 6.0×10¹² at/cm² to 4.0×10¹⁴ at/cm², and so forth. The Ns is preferably within the range of from 3.0×10¹² at/cm² to 8.0×10¹⁴ at/cm², more preferably from 3.0×10¹² at/cm² to 4.0×10¹⁴ at/cm², further preferably from 6.0×10¹² at/cm² to 2.0×10¹⁴ at/cm². The method for determining the Ns is given in the examples section.

It is preferable that at least a part of the noble metal has a specific oxidation state X. The specific oxidation state X of the noble metal may result from strong interaction with the metal oxide in the support. When the noble metal is Pt or Pd, the specific oxidation state X is 2+. When the noble metal is Rh or Au, the specific oxidation state X is 3+. A relative intensity of the noble metal having the specific oxidation state with respect to the total intensity of the noble metal of all oxidation states as measured by X-ray photoelectron spectroscopy (XPS) is preferably 50% or more, more preferably 75% or more, further preferably 80% or more, particularly preferably 90% or more. For example, if the noble metal is Pt, the relative intensity of Pt²⁺ with respect to the total intensity of Pt⁰, Pt²⁺ and Pt⁴⁺ (i.e. all oxidation states observed) is to be determined from the Pt 4f XPS spectrum. The detailed determination method is given in the examples section.

It is preferable that an infrared reflection-absorption spectrum (IRAS) measured on the catalytically active material shows an absorption band within the range of 2085 cm⁻¹ to 2120 cm⁻¹ that does not change upon heating the material from 300 K to 500 K, when measured under the following condition:
(1) exposing the catalytically active material in a reaction cell to a reaction mixture consisting of 1% CO and 5% O₂ (balanced by Ar) at 1 bar,
(2) heating the catalytically active material to 500 K at a rate of 60 K/min and reacting the mixture for 5 min at 1 bar at 500 K,
(3) cooling down the reaction mixture and the catalytically active material to 300 K, (4) removing the reaction mixture from the reaction cell and applying ultrahigh vacuum (UHV) conditions of 10⁻⁹ mbar or less,
(5) recording a spectrum in UHV at 300 K without additional exposure to CO,
(6) increasing the temperature to 500 K at a rate of 60 K/min and recording spectra at 320 K, 360 K, 400 K, 440 K, 480 K and 500 K, and
(7) comparing the absorption maxima in the spectra recorded at the respective temperatures.

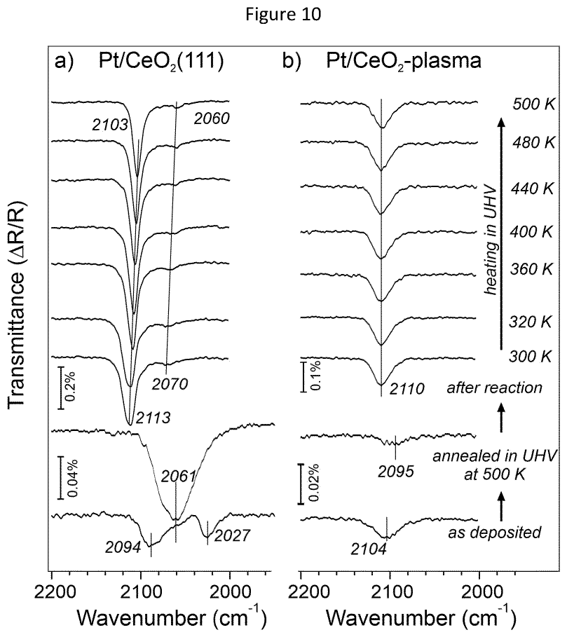

For any catalytically active material of the present invention, a corresponding absorption band within the range of 2085 cm⁻¹ to 2120 cm⁻¹ exists and can be easily determined by the person skilled in the art. In the case of Pt/CeO₂, the corresponding band (i.e. its absorption maximum) lies at 2110 cm⁻¹ ±1 cm⁻¹ as seen from FIG. 10.

According to the first embodiment of the present invention, the support is present in the form of particles and may e.g. form a powder. The shape of these particles is not particularly limited, and may be selected from ellipsoidal, cubic and spherical shapes.

According to the first embodiment of the present invention, the average particle diameter of the support is not limited, and preferably 10 nm to 1 μm, more preferably 10 nm to 500 nm, further preferably 10 nm to 100 nm, particularly preferably 10 nm to 50 nm, most preferably 10 nm to 30 nm. The average particle diameter of the support can be determined by STEM by measuring diameters of 20 arbitrarily selected particles and calculating the average thereof, as explained in the experimental section. As “diameter” the longest visible axis of the particle is taken. If the accuracy of the measurement is to be further increased, the measurement can be conducted with 100 arbitrarily selected particles. If several particles form together agglomerates, the diameter of the primary particles is taken for the measurement.

According to the first embodiment of the present invention, the support preferably consists of the metal oxide selected from TiO₂, CeO₂, ZnO, SnO₂, Ga₂O₃, In₂O₃, ZrO₂, and Fe₂O₃.

According to one preferred aspect of the first embodiment of the present invention, the catalytically active material preferably consists of the metal oxide support and the noble metal which is at least in part atomically dispersed. According to one further preferred aspect of the first embodiment of the present invention, the catalytically active material preferably consists of the metal oxide support and the noble metal of which 50% or more, 75% or more, 80% or more, or 90% or more is atomically dispersed.

XPS can be used to measure the percentage of the atomically dispersed noble metals. In particular, the relative intensity of the noble metal having the specific oxidation state X with respect to the total intensity of the noble metal of all oxidation states as described above can be taken as feature indicating the percentage of the atomically dispersed noble metals.

For example, when the noble metal is Pt, the percentage of the atomically dispersed Pt can be determined from the relative intensity of Pt²⁺ with respect to the total intensity of Pt of all oxidation states. Pt⁰ and Pt⁴⁺ may be attributed to clusters of metallic Pt and PtO₂, respectively.

STEM can also be used to measure the percentage of the atomically dispersed noble metals. In particular, 100 STEM pictures (magnification to e.g. 10 nm×10 nm) clearly showing noble metal species (atomically dispersed noble metals and clusters containing two or more noble metals) are arbitrarily selected, and the percentage of the atomically dispersed noble metals is determined for each STEM picture and then averaged. In STEM pictures, the noble metal species may appear brighter than the support.

The percentage of the atomically dispersed noble metals is preferably determined by XPS.

According to the first embodiment of the present invention, the surface density Ns of the noble metal is preferably 3.0×10¹² at/cm² to 4.0×10¹⁴ at/cm², more preferably from 3.0×10¹² at/cm² to 2.0×10¹⁴ at/cm², further preferably 3.0×10¹² at/cm² to 1.0×10¹⁴ at/cm², even further preferably 6.0×10¹² at/cm² to 1.0×10¹⁴ at/cm².

According to the second embodiment of the present invention, the support is in the form of a film. The film support may be provided on a base material. In one embodiment the base material provides a planar area on which the film support can be grown, e.g. by vapor deposition of the metal component of the reducible oxide on an oxidized surface of the base material. The base material is not particularly limited, as long as it does not adversely affect the catalytically active material. For example, a base material made of a metal, an alloy or a ceramic may be used. Examples of the metal used as the base material include, but are not limited to, Ru, Cu, Al, and the like. The metal used as the base material may be a single crystal, and examples thereof include a Ru(0001) single crystal and a Cu(111) single crystal. Examples of the alloy used as the base material include, but are not limited to, steel, brass, and the like. Examples of the ceramic used as the base material include, but are not limited to, alumina, silica, silicon carbide, and the like. The skilled person is able to select the base material suitable for the reaction conditions to which the catalytically active material is exposed.

According to the second embodiment of the present invention, the thickness of the film support is not particularly limited, and preferably 5 nm to 50 nm, more preferably 10 nm to 20 nm. The thickness of the metal oxide film can be determined by, for example, XPS by measuring the attenuation of an XPS signal of the base material, on which the metal oxide film is provided. For instance, if the metal oxide film is provided on a Ru base material, the attenuation of Ru 3d signal in the XPS spectra can be measured to determine the thickness of the metal oxide film support. Likewise, if a Cu base material is used, the attenuation of Cu 2p signal in the XPS spectra can be measured. Likewise, if a steel base material is used, the attenuation of Fe 2p signal in the XPS spectra can be measured. The skilled person is able to appropriately select the signal resulting from the element in the base material and measure the attenuation thereof to determine the thickness of the metal oxide film. The details of the measurement method are given in the examples section.

According to the second embodiment of the present invention, the surface density Ns of the noble metal is preferably 3.0×10¹² at/cm² to 4.0×10¹⁴ at/cm², more preferably from 6.0×10¹² at/cm² to 4.0×10¹⁴ at/cm², further preferably 1.2×10¹³ at/cm² to 2.0×10¹⁴ at/cm².

According to the second embodiment of the present invention, the support may consist of a substrate and clusters supported on the substrate, the substrate and the clusters comprising the same metal oxide. Preferably, the substrate and the clusters consist of the same metal oxide. The average diameter of the clusters may be 2.0 nm or less, preferably 1.5 nm or less as determined by STM by plotting a topography profile along a straight line corresponding to the length of 10 nm; measuring the distance between two adjacent minima; multiplying the measured value by a correction factor of 0.5 and recording the obtained value as a diameter; repeating the measurement on 20 arbitrarily selected surface areas showing a coverage by metal oxide clusters; and calculating the average value. The detailed method is given in the examples section.

The number of the metal oxide clusters on the metal oxide substrate and normalized to the surface area of the metal oxide substrate (surface cluster density) is not particularly limited.

The surface cluster density is preferably at least 0.05 clusters/nm², more preferably at least 0.08 clusters/nm², further preferably at least 0.10 clusters/nm², and most preferably at least 0.12 clusters/nm². The surface cluster density can be determined by STM by counting the number of the metal oxide clusters present within an arbitrarily selected area. The detailed method is given in the examples section.

According to one preferred aspect of the second embodiment of the present invention, the catalytically active material preferably comprises the noble metal of which e.g. 50% or more, 75% or more, 80% or more, or 90% or more is atomically dispersed. The percentage of the atomically dispersed noble metals can be determined as described above for the first embodiment.

The catalytically active material of the present invention is particularly suitable for the oxidation of CO and selective hydrogenation of alkynes (e.g. acetylene hydrogenation). The catalytically active material of the present invention may also be suitable for other catalytic reactions, including for instance water-gas-shift reaction, steam reforming and oxidation of natural gas.

The catalytically active material of the present invention shows an excellent stability and activity which is also reflected by suitably low onset reaction temperatures in the target reactions such as CO oxidation and selective alkyne hydrogenation. At least in part, these properties seem to be related to the atomic dispersion of noble metals on the surface of metal oxide clusters. The available experimental evidence further indicates that the uniformity of this atomic dispersion and/or the noble metal loading (in at/cm²) can be enhanced by the inventive methods for producing the catalytically active material as follows.

### II. Method for Producing the Catalytically Active Material

A method for producing the catalytically active material according to the first embodiment of the present invention may comprise the following steps:
(1) providing a precursor for the catalytically active material comprising a support in the form of particles comprising a metal oxide and noble metal on the surface of the support,
(2) calcining the precursor for the catalytically active material in the presence of O₂, and
(3) carrying out a non-thermal plasma treatment of the calcined precursor for the catalytically active material in the presence of O₂.

At the end of step (1), the noble metal may be present on the surface of the support in elementary form (oxidation state=0) or as noble metal compound, e.g. as noble metal salt.

The method for providing the precursor for the catalytically active material is not particularly limited, and the step (1) is preferably selected from the following steps (1a) and (1b):
(1a) depositing the noble metal onto the support, and
(1b) co-precipitating the noble metal and the metal oxide from an aqueous solution containing salts of the noble metal and the metal oxide.

The method for depositing the noble metal onto the support according to the step (1a) is not particularly limited, and examples thereof include physical vapor deposition, chemical vapor deposition and deposition from an aqueous solution of a salt of the noble metal. It is preferred that the noble metal is deposited from the aqueous solution of a salt of the noble metal onto the support, and known methods such as strong electrostatic adsorption (SEA) and impregnation may be appropriately used. The deposition is preferably conducted such that the noble metal deposition is controllable in terms of the surface density of metal atoms on the support. In step (1a), the support preferably consists of the metal oxide.

The salt of the noble metal is not particularly limited. Examples of the salt of the noble metal include, but are not limited to, tetraammineplatinum(II) nitrate, tetraammineplatinum(II) chloride, platinum(II) acetylacetonate; tetraamminepaladium(II) nitrate, tetraamminepaladium(II) chloride; rhodium(III) acetylacetonate; hydrogen tetrachloroaurate(III), gold(III)-chloride, to name few.

As used herein, the expressions “calcining” or “calcination” refers to a thermal treatment of a solid at an elevated temperature in the presence of O₂ under dry conditions. As used herein, the expression “dry” indicates that no additional water is introduced to the atmosphere, in which the calcination is carried out. For example, the calcination in the atmospheric air is to be understood as being carried out under the dry conditions.

The calcination of the precursor for the catalytically active material is preferably carried out at a temperature of 573 K to 973 K, more preferably 623 K to 873 K, further preferably 673 K to 823 K. The calcination is preferably carried out at a concentration of O₂ of 5 vol. % or more, more preferably 10 vol. % or more. For example, air can be used as the source of O₂.

The non-thermal plasma treatment of the calcined precursor for the catalytically active material is carried out in the presence of O₂. Prior to ignition of the plasma, the pressure is preferably reduced to 40 mbar or less, more preferably 20 mbar or less. The pressure during the non-thermal plasma treatment is preferably 40 mbar or less, more preferably 20 mbar or less. The non-thermal plasma treatment is preferably carried out at a concentration of O₂ in an inert gas of 5 vol. % or more, more preferably 80% or more, further preferably 100% (pure oxygen). The inert gas may be selected from N₂, He, Ne, Ar, and the like.

The non-thermal plasma treatment may be carried out under a static atmosphere or under a gas flow, the latter being preferred. When the non-thermal plasma treatment is carried out under a gas flow, it is preferred that the calcined precursor for the catalytically active material is blown up (levitated) by the gas flow during the non-thermal plasma treatment.

The source of the non-thermal plasma is not limited, and examples include inductively coupled plasma, capacitively coupled plasma, DC glow discharge, microwave plasma, radio-frequency plasma, cold plasma jet, and dielectric barrier discharge plasma. The source of the non-thermal plasma is preferably microwave plasma or radio-frequency plasma.

When the source of the non-thermal plasma is a microwave plasma, an anode voltage of the plasma source is preferably 0.2 kV to 2 kV, and an emission current is preferably 0.1 μA to 1 μA.

When the source of the non-thermal plasma is the radio-frequency plasma, the power of the radio-frequency plasma generator is preferably 20 W to 300 W.

A duration of the non-thermal plasma treatment can be appropriately adjusted as desired, and is usually 1 min to 300 min, preferably 5 min to 180 min, more preferably 10 min to 120 min, further preferably 30 min to 90 min.

A method for producing the catalytically active material according to the second embodiment of the present invention may be selected from the following methods A and B.

Method A comprises the following steps:
(1) providing the metal oxide film support,
(2) carrying out a non-thermal plasma treatment in the presence of O₂ to obtain a plasma-treated metal oxide film support, and
(3) depositing the noble metal onto the plasma-treated metal oxide film support in gas phase, preferably by physical vapor deposition of noble metal atoms.

Method B comprises the following steps:
(1) providing the metal oxide film support,
(2) depositing the noble metal onto the metal oxide film support from a solution containing the noble metal, and
(3) carrying out a non-thermal plasma treatment in the presence of O₂.

The method for providing the metal oxide film support is not particularly limited, and any method available in the art may be used. The method for providing the metal oxide film may include molecular beam epitaxy, laser ablation, chemical vapor deposition, sol-gel method, and the like. In one embodiment, the metal oxide film is provided on a base material as explained before in connection with the second embodiment of the invention.

In step (2) of method A and in step (3) of method B, the non-thermal plasma treatment is carried out in the presence of O₂. The non-thermal plasma treatment is preferably carried out at a pressure of 1×10⁻⁶ mbar to 1×10⁻² mbar, more preferably 1×10⁻⁶ mbar to 1×10⁻⁴ mbar, further preferably 1×10⁻⁶ mbar to 1×10⁻⁵ mbar. The partial pressure of O₂ during the non-thermal plasma treatment is preferably 1×10⁻⁶ mbar to 1×10⁻² mbar, more preferably 1×10⁻⁶ mbar to 1×10⁻⁴ mbar, further preferably 1×10⁻⁶ mbar to 1×10⁻⁵ mbar. In one embodiment, the non-thermal plasma treatment is preferably carried out in 100% O₂ at the above-described pressure.

The non-thermal plasma treatment may be carried out under a static atmosphere or under a gas flow.

A source of the non-thermal plasma is not particularly limited, and examples include inductively coupled plasma, capacitively coupled plasma, DC glow discharge, microwave plasma, radio-frequency plasma, cold plasma jet, and dielectric barrier discharge plasma.

The source of the non-thermal plasma is preferably microwave plasma or radiofrequency plasma.

When the source of the non-thermal plasma is the microwave plasma, an anode voltage of the plasma source is preferably 0.2 kV to 2.0 kV, more preferably 0.5 kV to 1.5 kV, and an emission current is preferably 0.1 μA to 1.0 μA.

When the source of the non-thermal plasma is the radio-frequency plasma, the power of the radio-frequency plasma generator is preferably 20 W to 300 W.

The duration of the non-thermal plasma treatment can be appropriately adjusted as desired, and is usually 1 min to 300 min, preferably 5 min to 180 min, more preferably 10 min to 120 min, further preferably 30 min to 90 min.

According to step (3) of the method A, the method for depositing the noble metal onto the plasma-treated metal oxide film support in gas phase is not particularly limited, and examples thereof include physical vapor deposition and chemical vapor deposition, physical vapor deposition being preferred. According to step (2) of the method B, the method for depositing the noble metal onto the plasma-treated metal oxide film from a solution containing the noble metal is not particularly limited, and examples thereof include deposition from an aqueous solution of a noble metal salt as a precursor. When the noble metal is deposited from an aqueous solution, the methods and the materials described in the method for producing the catalytically active material according to the first embodiment of the present invention can be applied.

According to the method as described herein, it can be assumed that all the deposited noble metal is present at the surface of the metal oxide support and the metal oxide clusters.

Hence, a high catalytic activity of the catalytically active material can be achieved with a small amount of the noble metal.

### III. Catalytic Oxidation of CO

The catalytically active material of the present invention can be suitably used in catalytic oxidation of CO to CO₂ (also referred to as “catalytic oxidation”).

In the catalytic oxidation of CO to CO₂, a reaction gas mixture comprising CO and O₂ is reacted in the presence of the catalytically active material to produce CO₂. The pressure of the reaction gas mixture is not particularly limited and may be 0.8 bar to 2.0 bar, preferably 0.8 to 1.5 bar, more preferably 0.9 to 1.2 bar.

The reaction temperature for the catalytic oxidation is not particularly limited and may be 323 K to 573 K, preferably 353 K to 523 K, more preferably 353 K to 473 K.

The composition of the reaction gas mixture is not particularly limited, as long as it comprises CO and O₂. The content of CO in the reaction gas mixture may be, for instance, 5 vol. % or less, preferably 3 vol. % or less, more preferably 2 vol. % or less. The content of O₂ in the reaction gas mixture may be 2 vol. % or more and 25 vol. % or less, preferably 5 vol. % or more and 20 vol. % or less. The molar ratio CO/O₂ of the reaction gas mixture is preferably 2.0 or less, more preferably 1.0 or less, further preferably 0.5 or less, most preferably 0.2 or less.

### IV. Catalytic Hydrogenation of Alkyne

The catalytically active material of the present invention can be suitably used in catalytic hydrogenation of alkyne into alkene (also referred to as “catalytic hydrogenation”). The alkyne used in the catalytic hydrogenation is preferably a C₂-C₅ alkyne, more preferably a C₂-C₃ alkyne.

In the catalytic hydrogenation of alkyne into alkene, a reaction gas mixture comprising an alkyne and H₂ is reacted in the presence of the catalytically active material in gas phase to produce an alkene. For instance, when acetylene (ethyne) is used as the alkyne, ethylene (ethene) is produced as the product (alkene).

The pressure of the reaction gas mixture is not particularly limited, and may be 0.8 bar to 2.0 bar, preferably 0.8 to 1.5 bar, more preferably 0.9 to 1.2 bar.

The reaction temperature for the catalytic hydrogenation is not particularly limited and may be 323 K to 573 K, preferably 323 K to 523 K, more preferably 353 K to 473 K.

A composition of the reaction gas mixture is not particularly limited, as long as it comprises the alkyne and H₂. The content of the alkyne in the reaction gas mixture may be, for example, 10 vol. % or less, preferably 5 vol. % or less, more preferably 3 vol. % or less. The content of H₂ in the rection gas mixture may be, for example, 3 vol. % or more, 5 vol. % or more, and 10 vol. % or more. The molar ratio H₂/alkyne is preferably 1.0 or more, more preferably 2.0 or more.

### EXAMPLES

Herein below, the present invention will be described in more detail with reference to the examples. However, the present invention is not limited to the following Examples.

### Analytical Methods

Scanning tunneling microscopy (STM) analysis—average diameter and number of metal oxide clusters; and low-energy electron diffraction analysis (LEED)—surface ordering in film supports

The STM measurements were performed at a sample voltage of 3.8 V and a constant tunneling current of 30 pA.

To determine the average diameter of metal oxide clusters, such as CeO₂ clusters, a topography profile was plotted along a straight line corresponding to the length of 10 nm. In the topography profile, a distance between two adjacent minima was measured, and the measured value was multiplied by a correction factor of 0.5 to correct for tip/cluster convolution effect. The obtained corrected value was recorded as a diameter. The diameters were measured on 20 arbitrarily selected metal oxide clusters, and the average value was calculated.

To determine the surface density of metal oxide clusters, i.e. the number of metal oxide clusters normalized to the surface area of the metal oxide substrate, in the recorded STM images, the number of metal oxide clusters within an arbitrarily selected 100 nm×100 nm area was counted. This measurement was repeated in four different sample spots, i.e. the measurements were carried out in five different sample spots in total. The mean value was taken as “density of metal oxide clusters”.

The low-energy electron diffraction (LEED) analysis was carried on the same instrument, and the LEED patterns were recorded at 81 eV.

Scanning transmission electron microscopy (STEM) analysis—average diameter of metal oxide particles in powder

STEM images were recorded on the 200 kV JEOL JEM ARM200F probe/image-corrected TEM (JEOL Ltd.).

The average diameter of the metal oxide particles, e.g. CeO₂ particles, of the metal oxide powder support, was determined by measuring diameters of arbitrarily selected 20 particles and calculating the average thereof. In the event that the STEM image showed a non-spherical shape for the selected particle, the longest axis was taken as diameter.

X-ray photoelectron spectroscopy (XPS)—elemental composition in the catalytically active material (film) and chemical (oxidation) state of the elements at surface

The relative intensity of the noble metal having the specific oxidation state (X) with respect to the total intensity of the noble metal of all oxidation sates was measured by XPS. In a case where the noble metal is Pt, the specific oxidation state is 2+. Also, in case the catalytically active material is a film, the number of the noble metals per surface area of the catalytically active material Ns (at/cm²) was measured by XPS. XPS spectra were measured with a Phoibos 150 analyzer (SPECS GmbH) using an Al Kα X-ray source (hv=1486.6 eV). Spectral analysis (background subtraction and deconvolution) was performed with the CasaXPS software (available from Casa Software Ltd; version 2.3.18), and the measured spectrum was subjected to the background subtraction using a Shirley background. In the case of Pt/CeO₂ film samples Pt 4f, Ce 3d and O 1s core level spectra were recorded at pass energies of 25 eV, 50 eV and 50 eV, respectively. The skilled person is able to select core levels of target elements and attribute binding energies to oxidation states by using the common general knowledge. For instance, tabulated list of binding energies such as NIST X-ray Photoelectron Spectroscopy Database belong to the skilled person's common general knowledge.

The Pt spectrum was subjected to a spectral deconvolution analysis using a CasaXPS software. For each Pt spectrum, the background subtraction was carried out in the range of 68 eV to 80 eV. The spectra were deconvoluted using a Gaussian/Lorentzian line shape. The maximum value of full-width half maximum (FWHM) of each peak was set at 2.0 eV. The relative intensity of Pt²⁺ was calculated as the area percentage of the peak at 72.6 eV relative to the total peak area of the Pt 4f_{7/2} signal.

### Measurement of Metal Oxide Film Thickness

The thickness of the metal oxide film prepared on the base material, e.g. CeO₂ film deposited on the Ru(0001) single crystal, was determined by XPS from the attenuation of the strongest signal stemming from the base material, e.g. Ru 3d signal, compared to that of the base material prior to the deposition of CeO₂ film.

### Raman Spectroscopy—Identification of the Surface Peroxo Species

The Raman spectroscopy was carried out on inVia™ Raman Microscope (Renishaw) using a 532 nm excitation laser. For each sample, measurements were carried out on five different sample spots. At each sample spot, three scans were acquired, and average thereof was recorded. The acquisition time of each scan was 20 seconds. The spectra were normalized to the principal peak of the support (for instance, F₂g peak at 465 cm⁻¹ for CeO₂). The normalized spectra recorded at the five different sample spots were averaged.

### N₂ Physisorption Analysis (BET Specific Surface Area)

The specific surface area of the catalytically active material in particle form (powder) was determined by the BET method from the N₂ adsorption isotherm. The N₂ physisorption measurements were carried out on ASAP 2020 PLUS (Micromeritics Instrument Corporation).

Number (Ns) of Noble Metal Atoms Normalized to the Surface Area of the Catalytically Active Material (Particles) in at/Cm²

For the catalytically active material in particle form, the surface density of single atoms (i.e., the number (Ns) of the noble metal atoms normalized to the surface area of the catalytically active material) was calculated from the specific surface area (m²/g converted into cm²/g) and the amount (g converted into at) of the deposited noble metals. If the noble metal is deposited by an impregnation technique, the total amount (in g) of noble metal atoms present in the aqueous solution can be used for the calculation if the deposition was quantitative. Otherwise, the amount follows from the yield achieved.

### Calculation of O 1s Binding Energy

The binding energies of the O 1s level at the surface were calculated by using density functional theory (DFT) within the final-state approximation and reference to the O 1s signal oxygen in a ceria bulk (2×2×2) supercell. The details for the DFT are given in a later section.

### CO Oxidation Reaction on the Catalytically Active Material in the Film Form

The reactivity measurements were performed in a high-pressure cell (SPECS HPC 20) connected to the main UHV analysis chamber. The catalytically active material was heated by a halogen lamp outside of the chamber through a quartz window. The reaction mixture consisted of 10 mbar of CO, 50 mbar of O₂ and balanced by He to 1 bar. The gas composition in the reactor was analyzed by gas leaking through a quartz micro-capillary into a quadrupole mass spectrometer (QMS, MKS Instruments). Starting from room temperature, the temperature of the catalytically active material was increased stepwise with a 50 K increment, and the CO₂ production was monitored by QMS. In experiments aimed at testing the stability of the atomically dispersed noble metal in the catalytically active material in the CO oxidation reaction, the sample was heated to 523 K with a rate of 1 K/s, and kept under reaction conditions for 10 min before the sample was cooled down to 300 K and evacuated, and then transferred to the analysis chamber for post-characterization.

### Infrared Reflection-Absorption Spectroscopy (IRAS)

The IRAS measurements were performed on the catalytically active material in the film form with a Bruker 66 ivs FTIR spectrometer in an UHV chamber hosting a “high-pressure” cell (reaction volume 1 I) for exposing the samples to gas mixtures at near atmospheric pressures.

The IRAS spectra of CO adsorption were measured following the steps described below:
(1) cooling down the catalytically active material to 200 K in UHV,
(2) dosing CO to the pressure of 10⁻⁶ mbar for 100 s and subsequent pumping CO out down to 10⁻⁹ mbar or below,
(3) recording an IRAS spectrum
(4) heating the catalytically active material to 500 K in UHV at a rate of 60 K/min and cooling down to 200 K, and
(5) repeating the steps (2) to (4) two times.

The IRAS measurements were performed also on the catalytically active material after exposure to a reaction mixture at 1 bar consisting of 1% CO and 5% O₂ balanced by Ar.

The measurement was carried out as follows:
(1) exposing the catalytically active material in a reaction cell to the reaction mixture,
(2) heating the catalytically active material to 500 K at a rate of 60 K/min and reacting the mixture for 5 min at 500 K,
(3) cooling down the catalytically active material to 300 K,
(4) removing the reaction mixture from the reaction cell and applying UHV conditions of 10⁻⁹ mbar or less,
(5) recording a spectrum in the UHV at 300 K without additional exposure to CO, (6) increasing the temperature of the catalytically active material to 500 K at a heating rate of 60 K/min and recording the spectra at 320 K, 360 K, 400 K, 440 K, 480 K and 500 K.

### CO Oxidation Reaction Over a Catalytically Active Material in Particle Form

The reactivity of the catalytically active material (powder) in the CO oxidation reaction was measured in a tubular packed-bed reactor. The gas phase composition was analyzed with a QMS (Hiden 20). The catalytically active material was sieved to below 75 μm, and 1 part by weight of the catalytically active material was physically mixed with 4 parts by mass of silica gel. The obtained mixture (5 parts by mass) was then mechanically mixed with 1 part by mass of acid-purified SiO₂ and loaded into the glass tube reactor. The CO oxidation reaction rate was measured at steady state in the mixture of 1% CO and 20% O₂, (He balance) at 1 bar at temperatures of 373 K, 383 K, 403 K, 423 K, 443 K and 473 K, increased stepwise, at least for 1 h at each temperature.

### Alkyne Hydrogenation Reaction on the Catalytically Active Material in the Particle Form

The reactivity of the catalytically active material in the particle form (powder) in the alkyne hydrogenation reaction was measured in a tubular packed-bed reactor, using acetylene as the alkyne. The gas phase composition was analyzed with a QMS (Hiden 20). The catalytically active material was sieved to below 75 μm and loaded into the glass tube reactor without dilution. The alkyne hydrogenation rate was measured at steady state in a mixture of 1% C₂H₂ and 5% H₂ (Ar balance) at 1 bar at temperatures of 373, 393, 413, 433, 453 and 473 K, increased stepwise, for 12 h at each temperature. The average ethene production rate observed during the hydrogenation reaction, for 12 h at each temperature, was recorded as the hydrogenation rate of acetylene.

### Pt/CeO₂ Film Samples

### Reference Example 1: Preparation of CeO₂(111) Film Support

The experiments were performed in an ultrahigh vacuum (UHV) chamber equipped with low energy electron diffraction (LEED), x-ray photoelectron spectroscopy (XPS), and scanning tunneling microscopy (STM), all from SPECS GmbH. The stoichiometric well-ordered CeO₂(111) films were grown on a Ru(0001) single crystal as follows. The Ru(0001) single crystal (9 mm in diameter, 1.5 mm in thickness, from MaTeck GmbH) was mounted onto a stainless-steel sample holder having a hole of 9 mm in diameter for heating the sample from the backside using an electron beam from a W filament. A type K thermocouple was spot-welded to the edge of the crystal. The surface of the Ru(0001) was oxidized in 10⁻⁶ mbar of O₂ at 1000 K. Ce was vapor-deposited onto the oxidized Ru(0001) surface using an electron beam assisted evaporator (Focus EMT3) from a Mo crucible filled with Ce (99.9%, Sigma Aldrich) in 10⁻⁶ mbar of O₂ at 90 K in amounts equivalent to form 4-5 monolayers (MLs) of CeO₂(111). Subsequently, the temperature of the Ru(0001) was increased at a rate of 1 K/s, and kept at 673 K during the deposition of further CeO₂ layers. The sample was then oxidized at 1000 K in 10⁻⁶ mbar of O₂. The thickness of the prepared CeO₂(111) film was about 5 nm. The prepared sample is denoted as “CeO₂(111)”.

### Reference Example 2: Preparation of CeO₂-Red Film Support

The reduced CeO₂₋ₓ(111) surface (henceforth referred to as “CeO₂-red”) was prepared by UHV annealing of the CeO₂(111) film at 1200 K for 5 min.

### Reference Example 3: Preparation of CeO₂-Step Film Support

The CeO₂(111) surface enriched with monoatomic steps (denoted as “CeO₂-step”) was prepared following the procedure described in detail in Nat. Commun. 7 (2016) 10801. In particular, it was obtained by depositing 0.3 monolayer (ML) of Ce onto the CeO₂(111) film and subsequent oxidation at 673 K. This resulted in the formation of small ML-high islands. This homoepitaxy of CeO₂ on CeO₂ yields clearly arranged samples with high step density.

### Reference Example 4: Preparation of CeO₂-Plasma Film Support

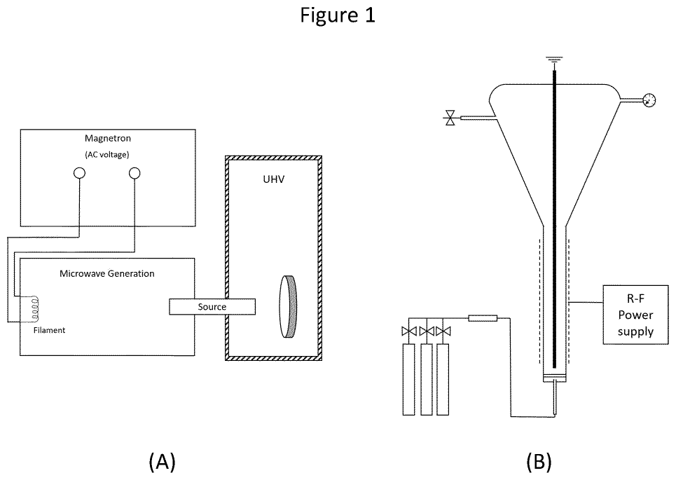

The oxygen plasma treatment of the CeO₂(111) films was carried out with a microwave plasma generator with a commercial plasma source (OSPrey, from Oxford Scientific, FIG. 1A) operated in 7×10⁻⁶ mbar of O₂ at an anode voltage of 1 kV and an emission current of 0.5 μA. The prepared sample is denoted as “CeO₂-plasma”.

### Example 1: Preparation of Pt/CeO₂-Plasma Film Sample

Pt was deposited onto the CeO₂-plasma film using an electron beam-assisted evaporator (Omicron EMT3) from a Pt rod. In order to minimize metal aggregation during the deposition at room temperature, Pt was deliberately deposited at low metal flux, i.e., 0.03 ML/min, as determined by a quartz microbalance (McVac), where 1.0 ML of Pt corresponds to one Pt atom per CeO₂(111) surface unit cell, i.e., 7.9×10¹⁴ Pt atoms/cm². During the deposition, the sample was biased at the same potential as the Pt rod. The amount of Pt was controlled to be 0.20 ML by XPS through analysis of the Pt 4f signal intensity. The obtained sample is denoted as “Pt/CeO₂-plasma”.

Subsequently an UHV annealing was applied to the obtained “Pt/CeO₂-plasma” sample. The sample was exposed to UHV and heated to 523 K at a heating rate of 120 K/min and kept at 523 K for 5 min in UHV, and then cooled down at a cooling rate of 60 K/min. The obtained sample is denoted as “annealed-Pt/CeO₂-plasma”.

The resulting annealed-Pt/CeO₂-plasma film catalyst showed the following features. The average diameter of the CeO₂ clusters was about 1.0 nm, as measured by STM. The number of Pt atoms at the surface of the sample (surface density, Ns) was 1.6×10¹⁴ at/cm² as calculated from the amount of Pt deposited (0.2 ML). The relative intensity of Pt²⁺ with respect to the total intensity of Pt⁰, Pt²⁺ and Pt⁴⁺ in the catalytically active material was 100%, as measured by XPS. By STM measurements, the number of the CeO₂ clusters normalized to the surface area of the CeO₂ substrate was determined to be 0.15 clusters/nm².

### Comparative Example 1: Preparation of Pt/CeO₂(111) Film Sample

Pt was deposited onto the CeO₂(111) film prepared in Reference Example 1 in the same manner as described in Example 1. The obtained sample is denoted as “Pt/CeO₂(111)”. The UHV annealing was applied to the Pt/CeO₂(111) in the same manner as in Example 1. The obtained sample is denoted as “annealed-Pt/CeO₂(111)”.

### Comparative Example 2: Preparation of Pt/CeO₂-Red Film Sample

Pt was deposited onto the CeO₂-red film prepared in Reference Example 1 in the same manner as described in Example 1. The obtained sample is denoted as “Pt/CeO₂-red”. The UHV annealing was applied to the Pt/CeO₂(111) in the same manner as in Example 1. The obtained sample is denoted as “annealed-Pt/CeO₂-red”.

### Comparative Example 3: Preparation of Pt/CeO₂-Step Film Sample

Pt was deposited onto the CeO₂-step film prepared in Reference Example 1 in the same manner as described in Example 1. The obtained sample is denoted as “Pt/CeO₂-step”. The UHV annealing was applied to the Pt/CeO₂(111) in the same manner as in Example 1. The obtained sample is denoted as “annealed-Pt/CeO₂-step”.

### Comparative Example 4: Preparation of Pt/CeO₂-Sputter Film Sample

First, the CeO₂(111) support was prepared in the same manner as described in Reference Example 1. Then, the surface thereof was roughened by bombarding the CeO₂(111) film with 1 keV Ar⁺ ions at 300 K for 5 min. The surface became considerably reduced as determined by XPS due to the preferential sputtering of lighter 0 atoms. To re-oxidize the ceria surface, the film was exposed to 10⁻⁶ mbar O₂ at 500 K. The resulting support is denoted as “CeO₂-sputter”.

Subsequently, deposition of 0.2 ML Pt onto this “CeO₂-sputter” support was carried out in the same manner as described in Example 1. The resulting sample is denoted as “Pt/CeO₂-sputter”. The UHV annealing was applied to the Pt/CeO₂(111) in the same manner as in Example 1. The obtained sample is denoted as “annealed-Pt/CeO₂-sputter”.

### Evaluation of the Results

The results obtained for the film samples are discussed in the following.

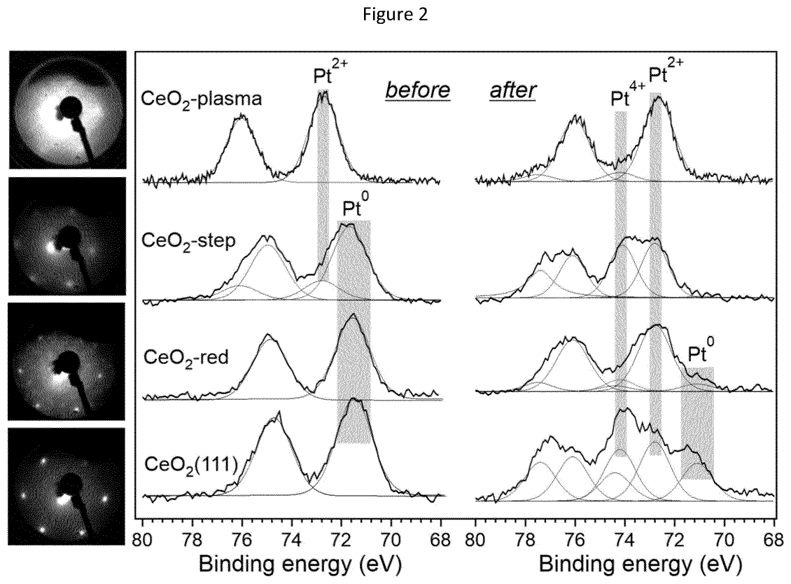

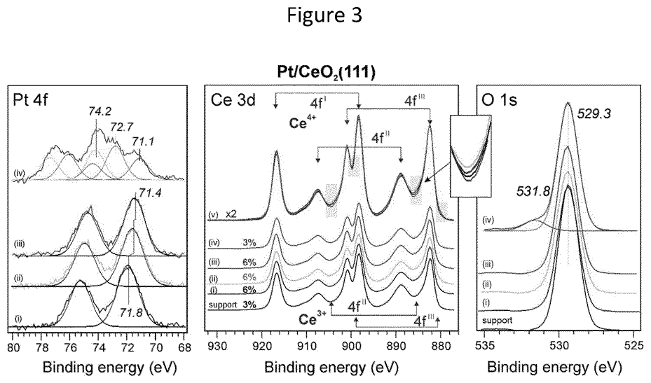

FIG. 2 show Pt 4f XPS spectra obtained for the (annealed-)Pt/CeO₂ samples of Example 1 and Comparative Examples 1-3 and measured before and after the previously described CO oxidation reaction at 523 K in the reaction mixture consisting of 10 mbar CO and 50 mbar O₂, balanced by He to 1 bar. All samples before the reaction were annealed in UHV at 523 K in order to discriminate solely thermal from reaction-induced effects. The full XPS data set for the Pt 4f, Ce 3d and O 1s levels are shown in FIGS. 3-6.

First, the results of Comparative Example 1 are discussed. As deposited Pt species in the Pt/CeO₂(111) were characterized by a binding energy (BE) of 71.8 eV (FIG. 3) indicating the formation of small Pt metallic clusters. The peak shifted to lower BE (71.4 eV) in the annealed-Pt/CeO₂(111) sample, indicating sintering of the Pt metallic clusters upon UHV annealing. The spectrum obtained after the CO oxidation reaction consisted of several states determined by deconvolution. The 71.1 eV state is associated with even larger metallic nanoparticles (NPs), suggesting further Pt sintering under reaction conditions. Two other signals centered at 74.2 and 72.7 eV were assigned to Pt in the 4+ and 2+ oxidation states, respectively.

Analysis of the Ce 3d and O 1s XPS spectra (FIG. 3) measured for the CeO₂(111) support according to Reference Example 1 and the Pt/CeO₂(111) sample according to Comparative Example 1 revealed no noticeable difference before and after the Pt deposition and measured at 300 K. The relative amount of Ce³⁺ slightly increased in the annealed-Pt/CeO₂(111) sample compared to the Pt/CeO₂(111), indicating a Pt-induced partial ceria reduction upon the UHV annealing at 523 K. The Ce 3d spectrum fully recovered after the CO oxidation reaction in an 0-rich atmosphere. The O 1s signal at 529.3 eV showed no additional states associated with adsorbates (typically, hydroxyls and carbonates) before and after the Pt deposition, namely in the CeO₂(111) support and in the Pt/CeO₂(111) sample, since the stoichiometric CeO₂(111) surface is essentially inert to residual gases in the UHV background at 300 K. However, a considerable signal at 531.8 eV appeared after the CO oxidation reaction, which can be attributed to adventitious CO₂ (and probably water) adsorption on the bare ceria support at “high” pressures.

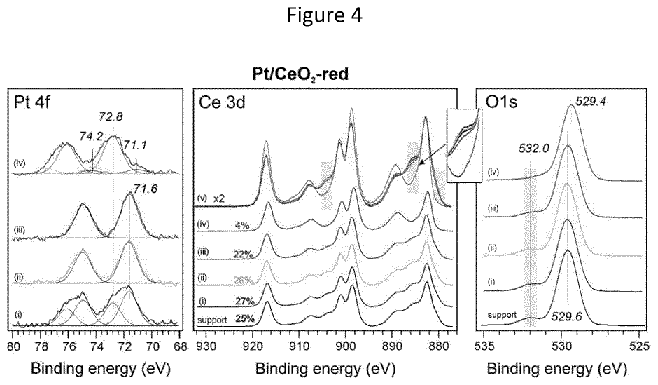

As seen from FIG. 2, the surface of the CeO₂-red film support of Reference Example 2 showed a complex (V7 x V7)-R19.1⁰ LEED pattern, which was assigned to long-range ordering of the 0 vacancies at the surface. In contrast to the stoichiometric CeO₂(111) support of Reference Example 1, this surface readily reacted with traces of water in the UHV background causing an additional weak O 1s signal at 532.0 eV from surface hydroxyls (FIG. 4). In the Pt/CeO₂-red sample of Comparative Example 2, beyond the Pt metal atoms constituting small clusters (BE at 71.6 eV), some Pt atoms were in the 2+ oxidation state (at 72.8 eV). However, in the annealed-Pt/CeO₂-red sample, the Pt²⁺ signal disappeared upon UHV annealing whereas the Pt⁰ signal gained in intensity compared to the Pt/CeO₂-red, so that the spectrum became virtually identical to the one observed on the stoichiometric CeO₂ surface (FIG. 2). After the CO oxidation reaction, Pt in the annealed-Pt/CeO₂-red sample was almost exclusively in the Pt²⁺ state (72.8 eV), with some minor contribution of Pt⁴⁺ (74.2 eV) and the Pt⁰ state shifted to 71.1 eV. Again, the results can be explained by the formation of relatively large Pt NPs which underwent oxidation. Concomitantly, the ceria surface became fully oxidized (no Ce³⁺ detected) because of the O₂-rich mixture used in the CO oxidation reaction (FIG. 4).

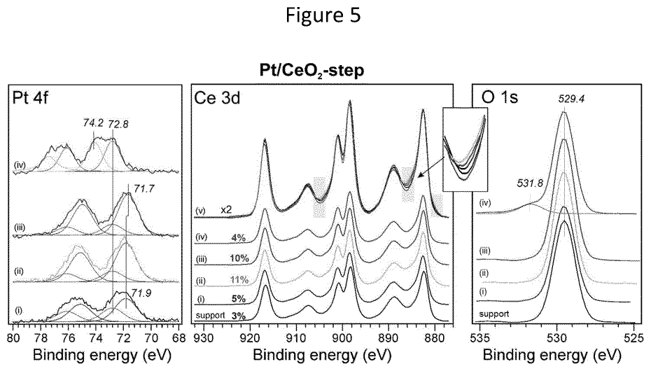

Turning to Comparative Example 3, in the Pt/CeO₂-step sample, the Pt atoms were partially in the 2+ and metallic states as deposited (BEs at 72.8 and 71.9 eV, respectively, see FIG. 5). In contrast to Comparative Example 1, after the UHV annealing, in the annealed-Pt/CeO₂-step sample, Pt²⁺ species were thermally stable and remained at the surface in addition to small metallic clusters, which sintered upon annealing (the corresponding BE shifted from 71.9 to 71.7 eV). After the CO oxidation reaction, no metallic Pt atoms were found on the surface, which contained only Pt²⁺ and Pt⁴⁺ species.

As shown above, in Comparative Examples 1-3, Pt deposits always formed Pt NPs upon UHV 5 annealing. A certain amount of thermally stable Pt²⁺ species found in the annealed-Pt/CeO₂-step sample can be explained by a strong adsorption of Pt single atoms at step edges. When exposed to the CO oxidation atmosphere, metal clusters became oxidized, thus giving rise to the 4+ and 2+ states, albeit their ratio depended on the initial particle size. The formation of Pt⁴⁺ and Pt²⁺ species on the Pt NPs in oxidizing atmosphere is well-documented in the literature, and is commonly attributed to PtO₂/PtO clusters or a thin PtOₓ oxide film on a large Pt NPs.

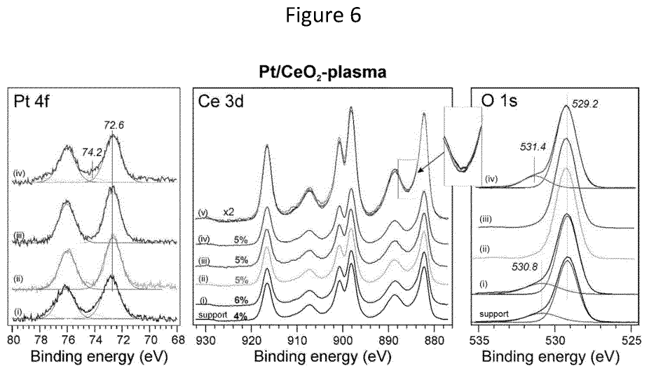

Finally, FIG. 2 shows the LEED pattern obtained for the CeO₂-plasma film support according to Reference Example 4. This sample substantially differed from the samples of Reference Examples 1-3 in that no long-range ordering was observed. As seen from Pt 4f SPX spectra of the Pt/CeO₂-plasma sample, starting from the “as deposited” sample, Pt remained exclusively in the 2+ state even in the CO oxidation reaction at near atmospheric pressure (FIG. 2 and FIG. 6).

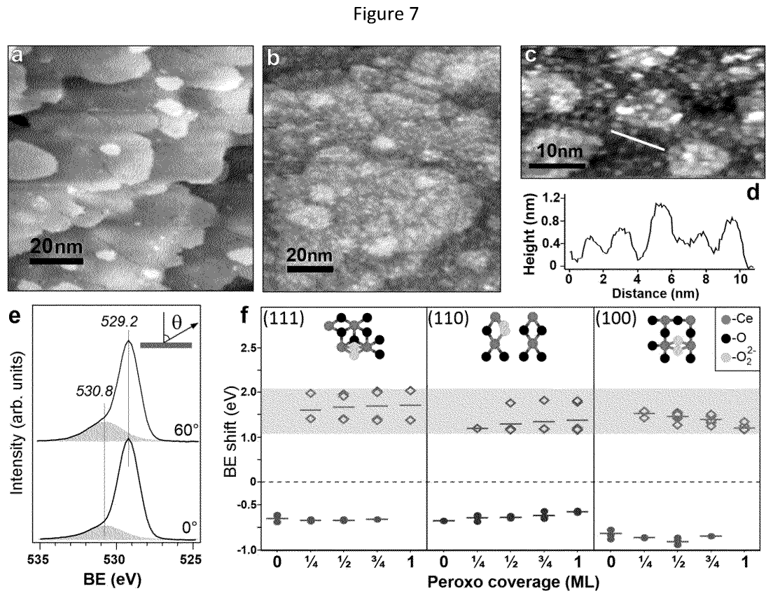

To shed light on the origin of the exceptional stability of Pt on the annealed-CeO₂-plasma surface, the film morphology was investigated by using STM. STM images (FIG. 7b-c) revealed the formation of CeO₂ clusters about 1.0 nm in diameter randomly distributed on the surface of the CeO₂-plasma support of Reference Example 4, so that the initial film morphology exhibiting terraces separated by monoatomic steps can still be recognized. Such CeO₂ clusters were not observed in the CeO₂(111) support of Reference Example 1 (FIG. 7a). The STM results also explain the lack of long-range order observed by LEED (see FIG. 2).

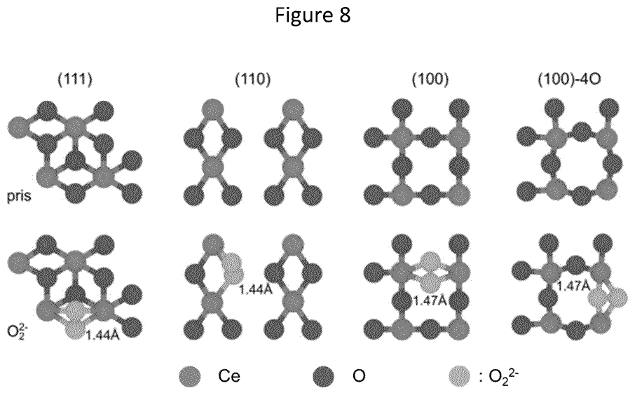

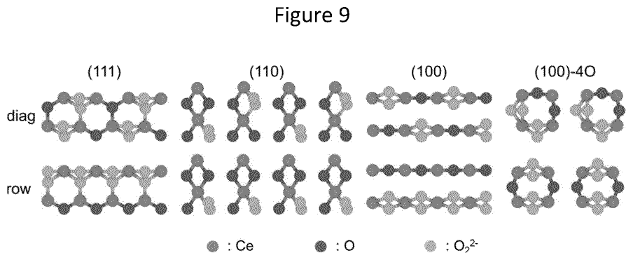

Importantly, the O 1s XPS spectra of the CeO₂-plasma support and the Pt/CeO₂-plasma sample (FIG. 7e) revealed a prominent shoulder at 530.8 eV, which cannot be assigned to adventitious hydroxyls and/or carbonates, showing a considerably higher BE of about 531.8 eV (FIG. 3-5). The signal is shifted by 1.6 eV with respect to the main peak at 529.2 eV and grew at increasing plasma exposure time. In addition, the signal gained in intensity when measured at more surface sensitive, grazing emission. All these findings point to the formation of new oxygen species which were absent on the other CeO₂ film supports employed in Comparative Examples 1-3. Based on DFT calculations of atomic 0 adsorption on the stoichiometric CeO₂(111) surface and corresponding BE shifts of the O 1s level (FIG. 7f, see details below), the 530.8 eV signal was assigned to surface peroxides, O₂²⁻ (FIG. 8-9). PBE+U core-level binding energies (in eV) for oxygen species on CeO₂ surfaces at varying peroxide coverages are presented in Table 1. The presented values correspond to the average results, as in each case, all of the oxygen atoms located in the surface layer were samples. The values are always referenced to the simulated O 1s signal of an oxygen atom in a ceria bulk (2×2×2) supercell. For 0.5 ML, two different geometric arrangements of the peroxide groups are possible (denoted as “diag” and “row” in FIG. 9).

TABLE 1

| | | Θ_{peroxide} [ML] | | | | | |
|---|---|---|---|---|---|---|---|
| Surface | Species | 0.0 | 0.25 | 0.5-diag | 0.5-row | 0.75 | 1.0 |
| (111) | O²⁻ | −0.81 | −0.85 | −0.84 | −0.85 | −0.82 | — |
| | O₂²⁻ | — | 1.59 | 1.67 | 1.64 | 1.68 | 1.70 |
| (110) | O²⁻ | −0.86 | −0.80 | −0.78 | −0.78 | −0.74 | −0.66 |
| | O₂²⁻ | — | 1.19 | 1.37 | 1.15 | 1.33 | 1.37 |
| (100) | O²⁻ | −1.14 | −1.23 | −1.39 | −1.26 | −1.19 | — |
| | O₂²⁻ | — | 1.52 | 1.47 | 1.43 | 1.39 | 1.19 |

To identify Pt species formed on the CeO₂-plasma films, IRA-spectroscopy was employed using CO as a probe molecule. The obtained CO IRAS results of the (annealed-)Pt/CeO₂-plasma sample of Example 1 and (annealed-)Pt/CeO₂(111) sample of Comparative Example 1 are discussed. The bare ceria surfaces were found not to adsorb CO under the conditions studied.

For the Pt/CeO₂(111) sample of Comparative Example 1, adsorption of CO (at 10⁻⁶ mbar) on Pt deposited onto a well-ordered CeO₂(111) surface at 200 K (FIG. 10a) resulted in IRAS bands centered at 2087 and 2024 cm⁻¹ and a weak band as a shoulder at around 2060 cm⁻¹. The former two signals disappeared after the UHV annealing at 500 K and then exposure to CO again, while the band at 2060 cm⁻¹ gained in intensity. The latter can be assigned to the stretching vibrations of CO adsorbed at the low-coordinated sites on metallic Pt nanoparticles located primarily at the step edges, as previously shown by STM. Accordingly, the 2087 and 2024 cm⁻¹ bands observed for the Pt/CeO₂(111) sample belong to CO adsorbed onto Pt clusters and aggregates with ill-defined structure, since its formation is affected by the limited diffusivity of surface ad-atoms at these relatively low temperatures.

In contrast, for the Pt/CeO₂-plasma sample of Example 1, CO adsorbed on the Pt species formed on the CeO₂-plasma surface showed a single band at 2104 cm⁻¹ after Pt deposition (FIG. 10b). The band was considerably reduced and red-shifted (to 2095 cm⁻¹) for CO adsorbed on the sample heated to 500 K in UHV. The spectra were fully reproducible after several thermal flashes to 500 K, indicating a good thermal stability of the Pt species, in full agreement with the XPS data. The IR band bore close similarity to those reported in the literature for powder Pt SACs proven by high resolution electron microscopy. However, the band disappeared after thermal flash to 700 K, indicating that the Pt atoms became inaccessible to CO. Note, however, that IRAS spectra on metal supported oxide films obey the selection rule such that only vibrations associated with dipole changes normal to the metal surface can be detected. Therefore, CO molecules oriented parallel to the Ru(0001) surface underneath the ceria film will be invisible in IRAS spectra.

These two systems (Example 1 and Comparative Example 1) were also investigated with IRAS after exposure to 1 bar of the reaction mixture consisting of 1% CO and 5% O₂ (balanced by Ar) in the “high-pressure” cell. After 5 min of reaction at 500 K and sample cooling to room temperature, the cell was pumped out and the IRAS spectra were recorded in UHV at 300 K without additional exposure to CO. Interestingly, the spectra revealed similar bands at 2110 cm⁻¹ in both systems (a weak and broad band at 2070 cm⁻¹ observed on annealed-Pt/CeO₂(111) can be assigned to traces of CO residing at the edges of metallic Pt NPs, see above). However, the spectra showed a different behavior on slow heating the sample to 500 K. On annealed-Pt/CeO₂(111) of Comparative Example 1 (see FIG. 10a), the 2113 cm-1 band gradually shifted to a lower wavenumber (by 10 cm⁻¹), and its integral intensity slightly decreased, indicating a partial desorption of CO. Such spectral changes and its peak position are characteristic for CO adsorbed on O-precovered Pt surfaces and can be explained in terms of the dipole-dipole interaction between neighboring CO molecules adsorbed on the partially oxidized surface of Pt NPs, which were initially formed prior to the reaction. The formation of relatively large metallic Pt NPs on CeO₂(111) can also explain the high signal intensity of the IRAS bands due to the image charge effects. In contrast, for the annealed-Pt/CeO₂-plasma of Example 1, a much weaker band at 2110 cm⁻¹ observed on the annealed-Pt/CeO₂-plasma surface stayed constant during sample heating to 500 K (FIG. 10b). Although this band fully disappeared upon heating to 700 K and could not be observed upon CO exposure at room temperature, it was reproduced after a second CO oxidation reaction run (not shown here). Based on these comparative XPS and IRAS studies, it can be concluded that Pt deposited onto the plasma-treated CeO₂ surface forms single atoms which remain stable in a reaction atmosphere, albeit its coordination to the ceria surface may be affected by the CO oxidation reaction.

Based on structural characterization of the CeO₂-plasma surface by LEED and STM, one may suggest that the enhanced stability of Pt originates from surface roughening that suppresses the diffusivity of the Pt ad-atoms and hence their aggregation. To examine this scenario, a rough CeO₂ surface was produced by bombarding a well-ordered CeO₂(111) film with 1 keV Ar⁺ ions at 300 K for 5 min. The obtained “CeO₂-sputter” support was employed to produce the (annealed-)Pt/CeO₂-sputter samples in Comparative Example 4.

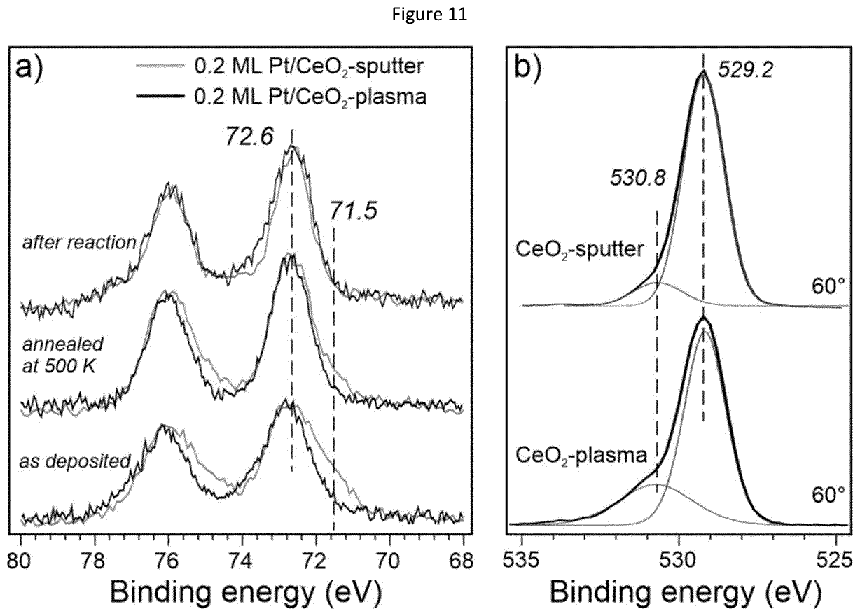

As seen from the Pt 4f XPS spectra (FIG. 11a), Pt in the Pt/CeO₂-sputter sample of Comparative Example 4 resulted in Pt species primarily in the 2+ state but a considerable amount of Pt was also found in the metallic state (at 71.5 eV), most likely as Pt NPs. A substantially higher intensity was observed for the 530.8 eV signal in the O 1s spectra of the Pt/CeO₂-plasma sample of Example 1 as compared to the Pt/CeO₂-sputter sample of Comparative Example 4, as shown in FIG. 11b. The results indicate that the plasma treatment of a CeO₂ support creates more “O-rich” sites to stabilize Pt single atoms.

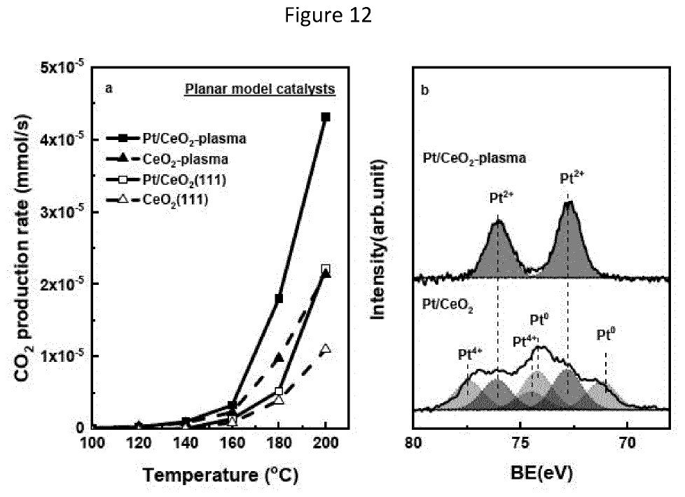

Finally, activity of the annealed-Pt/CeO₂-plasma sample of Example 1 in the CO oxidation reaction is compared with that of the annealed-Pt/CeO₂(111) sample of Comparative Example 1. Steady state CO₂ production rates were measured on the catalysts of Example 1 and Comparative Example 1 in a high-pressure cell filled with 10 mbar of CO and 50 mbar of O₂ balanced by He to 1 bar at different sample temperatures increased stepwise. The results are shown in FIG. 12a. As seen therefrom, the annealed-Pt/CeO₂-plasma sample of Example 1 showed a higher CO₂ production rate than the annealed-Pt/CeO₂(111) sample of Comparative Example 1.

### Pt/CeO₂ Powder Catalysts

### Reference Example 5: Preparation of Pristine CeO₂ Powder Support

The CeO₂ powder with high surface area (50 m²/g) were purchased from US Research Nanomaterials (Stock no. US3037) and used as received.

### Comparative Example 5: Preparation of Pristine Pt/CeO₂ Powder Catalyst

The powder Pt/CeO₂ sample was synthesized via the strong electrostatic adsorption (SEA) method. The high purity (99.995%) tetraammineplatinum(II) nitrate (TAPN) (Sigma Aldrich No. 482293) was used as the Pt precursor, and ammonium hydroxide (28%˜30% solution in water) was purchased from ACROS Organics. A pristine Pt/CeO₂ powder catalyst was obtained by the following steps.
1) 1 g of ceria was crushed, sieved to a powder finer than 75 μm, and then added to a solution of deionized water mixed with NH₄OH with a pH of 9 to prepare a support solution.
2) 10 mg of TAPN was dissolved in 5 mL of deionized water to obtain an aqueous TAPN solution, and 300 μL of the resulting solution was added to 25 mL NH₄OH solution with a pH of 9. The resulting solution (25.3 mL) was slowly added to the support solution via a burette, and this process took around 3.5 hours.
3) The solution obtained in above step 2) was dried in a rotary vacuum evaporator (343 K, 110 rpm).
4) The dried catalyst was calcined in a muffle oven at 723 K for 4 h in the air with a ramping temperature of 10 K/min and was then cooled down to the room temperature.

### Example 2: Preparation of Plasma-Treated Pt/CeO₂ Powder Catalyst

First, a pristine Pt/CeO₂ powder catalyst was prepared in the same manner as in Comparative Example 5. An O₂-plasma treatment was then carried out on the pristine Pt/CeO₂ powder catalyst in the following manner to obtain a plasma-treated Pt/CeO₂ powder sample.

The O₂-plasma treatment was carried out in a setup which consisted of a glass tube with frits, a funnel shape glassware, a mechanical pump, a radiofrequency plasma generator and a high voltage power supply. A schematic illustration of the setup for the O₂-plasma treatment is provided in FIG. 1B. The glass tube was wrapped with a Cu mesh which was connected to a high voltage power supply. The frits could hold the calcined powder while allowing the gas to blow from the bottom. The funnel-shaped glassware connected to the top of the tube and the other 3 outlets of the funnel-shaped glassware were connected to the pressure meter, mechanical pump and a tungsten bar for grounding. The tungsten bar extended to the bottom of the glass tube to achieve a uniformly distributed plasma.

The plasma was generated by a 20˜60 kHz high voltage power supply (PVM500). The peak voltage was measured by an oscilloscope with a high voltage probe and the power of the plasma was the product of the square root of the mean square (RMS) voltage and RMS current, which were measured by a multimeter. The power and frequency outputs of the powder supply were adjusted until a plasma with a desirable power was formed.

The conditions of the O₂ plasma treatment were as follows:
1) The pristine Pt/CeO₂ powder catalyst was introduced into the glass tube (between 100˜300 mg) and the tube was evacuated to below 20 mPa.
2) Oxygen was flowed from the bottom of the tube with a flow rate 20 mL/min O₂ set by a mass flow controller (MFC).
3) The powder sample started to levitate in a turbulent flow.
4) The plasma was ignited. The power output and frequency of the power supply was adjusted to achieve the target power of 200 W.
5) The plasma treatment took 1 hour.

### Reference Example 6: Preparation of Plasma-Treated CeO₂ Powder Support

A plasma-treated CeO₂ powder support was prepared by carrying out the O₂ plasma treatment in the same manner as in Example 2 to the CeO₂ nanoparticles of Reference Example 5.

### Examples 3 and 4

First, a pristine Pt/CeO₂ powder catalyst was prepared in the same manner as described in Comparative Example 5, with the sole difference that the amount of TAPN dissolved in 5 mL of deionized water in step 2) was varied as show below in Table 2. An O₂-plasma treatment was then carried out on the pristine Pt/CeO₂ powder catalyst in the same manner as in Example 2 to prepare a plasma-treated Pt/CeO₂ powder catalyst.

### Comparative Examples 6 and 7

A pristine Pt/CeO₂ powder catalyst was produced in the same manner as described for Comparative Example 5 with the sole difference that the amount of the TAPN solution added to the 25 mL NH₄OH solution in step 2) was varied in Comparative Examples 4 and 5 as shown below in Table 2. In Example 4 and Comparative Example 7, 20 mg of TAPN was dissolved in 10 mL of deionized water to prepare the aqueous TAPN solution.

TABLE 2

| | TAPN | | Pt/CeO₂ |
|---|---|---|---|
| Example No. | solution added | | (wt. %) |
| Example 2 | 300 | μL | 0.05 |
| Example 3 | 3.0 | mL | 0.5 |
| Example 4 | 6.0 | mL | 1.0 |
| Comparative | 300 | μL | 0.05 |
| Example 5 | | | |
| Comparative | 3.0 | mL | 0.5 |
| Example 6 | | | |
| Comparative | 6.0 | mL | 1.0 |
| Example 7 | | | |

Due to the impregnation preparation method used, it can be assumed that all the deposited Pt atoms are located on the surface of the CeO₂ support and the CeO₂ clusters in the prepared catalytically active materials. The surface density Ns of Pt atoms in the catalytically active materials are shown in Table 3.

TABLE 3

| | Pt/CeO₂ | Surface density |
|---|---|---|
| | (wt. %) | of Pt (at/cm²) |
| Example 2 | 0.05 | 3.1 × 10¹² |
| Example 3 | 0.5 | 3.1 × 10¹³ |
| Example 4 | 1.0 | 6.2 × 10¹³ |
| Comparative | 0.05 | 3.1 × 10¹² |
| Example 5 | | |
| Comparative | 0.5 | 3.1 × 10¹³ |
| Example 6 | | |
| Comparative | 1.0 | 6.2 × 10¹³ |
| Example 7 | | |

### Evaluation of the Results

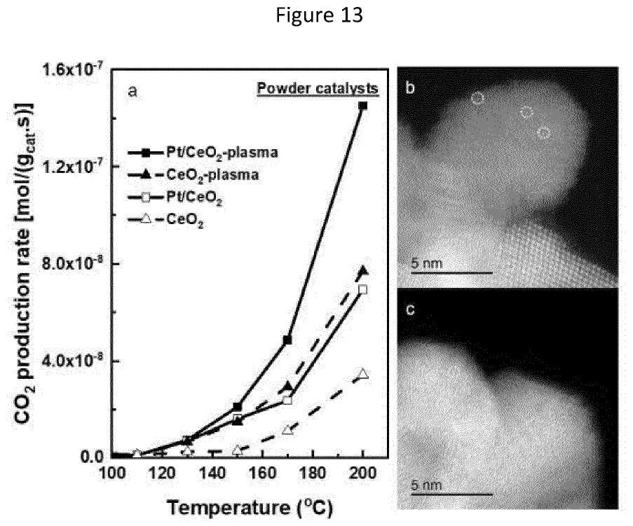

FIG. 13a shows CO₂ production rates measured for the Pt/CeO₂ powder samples of Example 2 and Comparative Example 5. As seen therefrom, the plasma-treated Pt/CeO₂ powder catalyst of Example 2 showed a higher CO₂ production rates than the pristine Pt/CeO₂ catalyst of Comparative Example 5.

FIGS. 13b and c show the STEM images of the plasma-treated Pt/CeO₂ catalyst of Example 2 recorded before and after the CO oxidation reaction at 523 K. The Pt single atoms were observed on the plasma treated sample before and after the CO oxidation reaction.

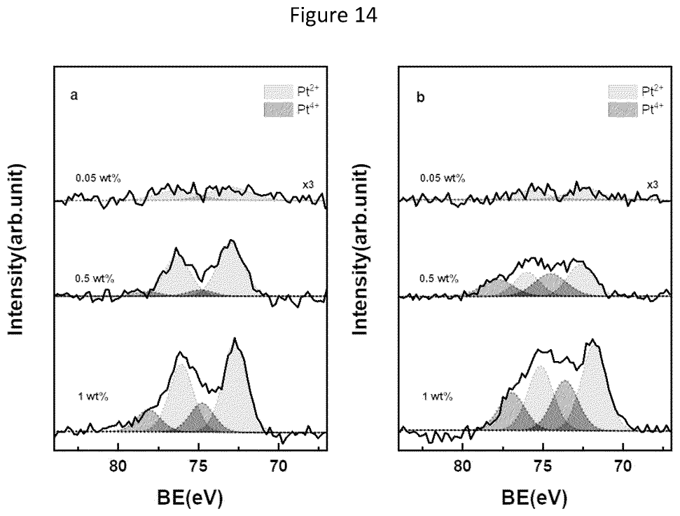

FIG. 14 shows Pt 4f XPS spectra of the Pt/CeO₂ powder catalysts of Examples 2-4 (FIG. 14a) and Comparative Examples 5-7 (FIG. 14b) measured after the CO oxidation reaction. At a Pt loading of 0.5 wt % or higher, both pristine and plasma-treated Pt/CeO₂ powder samples showed PtO₂ clusters, as indicated by the Pt⁴⁺ peaks in the XPS spectra. However, the content of the Pt⁴⁺ species was smaller on the plasma-treated Pt/CeO₂ catalysts (FIG. 14a) than those on the pristine Pt/CeO₂ catalysts representing the conventional synthesis (FIG. 14b), indicating a better atomic distribution of Pt.

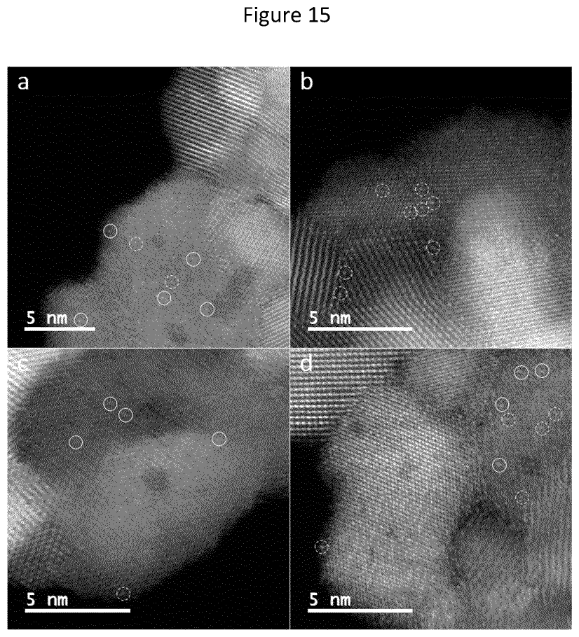

FIG. 15 shows STEM images of the plasma-treated Pt/CeO₂ catalysts of Examples 3 and 4 (FIGS. 15a and c) and the pristine Pt/CeO₂ catalysts of Comparative Examples 6 and 7 (FIGS. 15b and d) after exposure to the CO oxidation reaction. The pristine Pt/CeO₂ catalysts showed fewer Pt single atoms in favor of the formation of small oxidized clusters (PtO₂). It was difficult to detect Pt on CeO₂ on the pristine Pt/CeO₂ samples measured after CO oxidation as compared to the plasma-treated Pt/CeO₂ samples. Without being bound to the theory, it is postulated that this is due to the formation of PtO₂ clusters (in agreement with XPS) on the pristine Pt/CeO₂ catalysts. More Pt single atoms were found on the plasma-treated Pt/CeO₂ samples in STEM, suggesting a better distribution of Pt atoms, which is consistent with the XPS results.

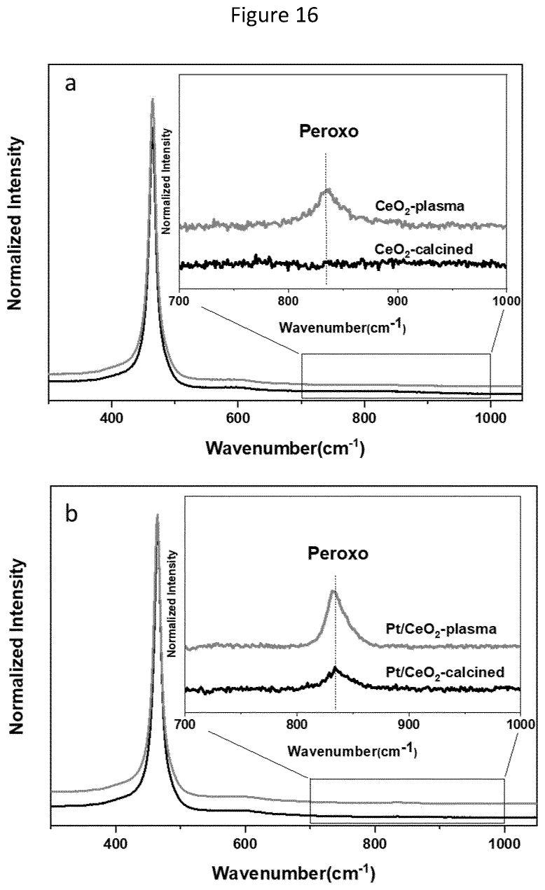

FIG. 16a shows Raman spectra of the pristine CeO₂ support of Reference Example 5 and the plasma-treated CeO₂ support of Reference Example 6. A Raman band at 465 cm⁻¹ observed in both spectra is attributed to the F₂g mode in bulk CeO₂. The plasma-treated CeO₂ support showed a Raman band at 831 cm⁻¹, while such a band was not observed for the pristine CeO₂ support. Without being bound to theory, it is postulated that the band at 831 cm⁻¹ is attributed to the surface peroxo (O₂²⁻) species.

FIG. 16b shows Raman spectra of the pristine Pt/CeO₂ catalyst of Comparative Example 4 and the plasma-treated Pt/CeO₂ catalyst of Example 2. Both Raman spectra are normalized to the principal band at 465 cm⁻¹ attributed to the F₂g mode. The plasma-treated Pt/CeO₂ catalyst showed an enhanced Raman band at 831 cm⁻¹, compared to that of the pristine Pt/CeO₂ catalyst. It is therefore postulated that the O₂ plasma-treatment on the Pt/CeO₂ catalyst enhances the formation of surface peroxo species.

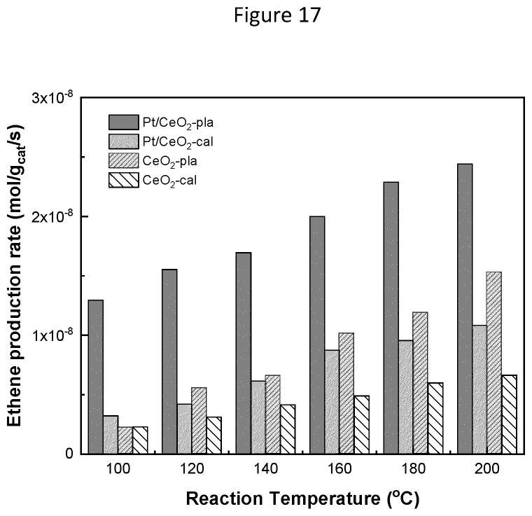

FIG. 17 shows ethene production rates measured for the Pt/CeO₂ powder samples of Example 2 and Comparative Example 5. As seen therefrom, the plasma-treated Pt/CeO₂ powder catalyst of Example 2 showed a higher ethene production rates than the pristine (calcined) Pt/CeO₂ powder catalyst of Comparative Example 5.

### Pt/TiO₂ Film Samples

### Reference Example 7: Preparation of TiO₂(110) Support

TiO₂(110) single crystal was purchased from SurfaceNet. The surface of the TiO₂(110) single crystal was cleaned by cycles of ion sputtering—oxygen annealing treatments, which are well known and documented in the literature (Diebold et. al., Surface Science, 1995, 331-333(PART B), 845-854), until no contamination was detected by XPS to obtain a clean TiO₂(110) single crystal.

### Reference Example 8: Preparation of TiO₂-Plasma Film Support

The oxygen plasma treatment of the clean TiO₂(110) single crystal of Reference Example 7 was carried out in the same manner as in Reference Example 4. The obtained sample is denoted as “TiO₂-plasma”.

### Example 5: Preparation of Pt/TiO₂-Plasma Film Sample

Pt/TiO₂-plasma sample was obtained in the same manner as in Example 1, except that Pt was deposited onto the TiO₂-plasma film support.

### Comparative Example 8: Preparation of Pt/TiO₂(110) Film Sample

Pt/TiO₂(110) sample was obtained in the same manner as in Example 5, except that Pt was deposited onto the clean TiO₂(110) film support. Accordingly, no plasma treatment was carried out.

### Evaluation of the Results

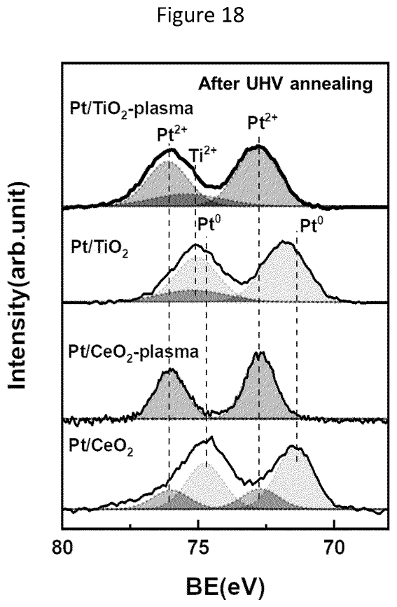

FIG. 18 shows Pt 4f XPS spectra obtained for the Pt/TiO₂ samples of Example 5 and Comparative Example 8. Pt in the Pt/TiO₂-plasma sample was almost exclusively in the 2+ oxidation state, as indicated by the XPS signal at 72.8 eV. This suggests that Pt in the Pt/TiO₂-plasma sample are present as single atoms. The Pt/TiO₂ sample showed a predominant signal at 71.6 eV, which is attributed to Pt⁰ and suggests the formation of clusters or nanoparticles of metallic Pt.

### Theoretical Model Studies

The studies presented herein describe mechanistic considerations in respect to one embodiment of the invention. They are not to be understood as describing the actual situation or as limiting the scope of the present invention but may serve to illustrate the same.

### Reference Example 9

### (Computational Method)

The calculations were performed with the Vienna Ab initio Simulation Package (VASP, version 5.4.4), employing the generalized gradient functional by Perdew, Burke and Ernzerhof (PBE). Core electrons were treated within the projector-augmented wave (PAW) method, while valence electrons were expanded in plane-waves with a basis set cut-off of 500 eV. An additional Hubbard correction (DFT+U) was applied to the Ce(4f) band following Dudarev et al. (Phys. Rev. B, 1998, 57, 1505-1509) using an on-site, effective U-parameter of 4.5 eV.

Optimization of bulk ceria yielded a theoretical lattice parameter of 5.491 Å. The (111), (110) and (100) surfaces were modelled using (2×2) slabs. Stepped surfaces were prepared accordingly as (1×2) supercells. At least 10 Å of vacuum were added on top of the slabs. The bottom layers of the slabs (approx. half of the total number) were kept fixed at the optimized bulk positions, while the upper layers were allowed to relax. A dipole correction along the surface normal was applied throughout. Sampling of the Brillouin zone was performed with G-centred k-point grids with a reciprocal grid spacing of about 0.025 Å⁻¹. Convergence of atomic positions was assumed when the absolute forces acting on each atom fell below 0.15 eV/A. Spin polarization was accounted for when necessary. Peroxide formation energies and adsorption energies of Pt and CO were obtained by stepwise structural relaxations, starting from the pre-optimized pristine surfaces. For the ceria NP/slab composite model, we employed an initial octahedral nanoparticle, following the well-known Wulff-construction and applied several cuts, ensuring CeO₂ stoichiometry. The resulting ceria nanoisland was anchored on a CeO₂(111)-(7×7) slab of nine atomic layers. The full system consisted of 178 cerium and 356 oxygen atoms (film —Ce₁₄₇O₂₉₄; NP —Ce₃₁O₆₂) and had a total expansion of 27.18×27.18×7.94 Å³. Due to the large cell dimensions, optimizations were carried out at Gamma-point.

Evaluation of CO vibrational frequencies was done by applying small, step-wise displacements of the relevant platinum, carbon and oxygen atoms (VASP-tag NFREE=2). Due to the high computational cost, a modified version of the NP/slab composite system, only keeping the surface layer of the underlying slab, was used and the Pt was kept fixed. Reference calculations have shown that deviations incurred by this approximation stay below 10 cm⁻¹, validating the approach. Additionally, in the case of the extended low-index surfaces, higher precision was applied by setting the VASP-tag PREC to Accurate. Lastly, all frequencies were scaled by referencing with the theoretical and experimental value of molecular CO (see Table 4). Table 4 shows the calculated energies (in eV) for Pt adsorption (referenced against gaseous single-atom Pt) on extended low-index ceria surfaces, CO-adsorption on single-atom Pt and corresponding CO vibrational frequencies (in cm⁻¹). The calculated vibrational frequencies were scaled using the scaling factor 1.008 based on the experimental (2143 cm⁻¹) and theoretical (2125 cm⁻¹) values obtained for CO in the gas phase.

TABLE 4

| | E_{ads}(Pt) | E_{ads}(CO) | v(CO) |
|---|---|---|---|
| Surface | [eV] | [eV] | [cm⁻¹] |
| (111) | −2.798 | −2.136 | 2093 |
| (111)-0.25 O₂²⁻ | −4.310 | −2.327 | 2081 |
| (111)-0.50 O₂²⁻ | −3.838 | −2.618 | 2080 |
| (row) | | | |
| (110) | −4.991 | −0.328 | 1961 |
| (100) | −5.282 | −2.136 | 2065 |
| (100)-4O | −6.445 | −0.575 | 2081 |

The plasma was modelled as isolated oxygen atoms (radicals). Binding energies of the O 1s level at surface were calculated within the final-state approximation and referenced to the O 1s signal of oxygen in a ceria bulk (2×2×2) supercell.

### DISCUSSION

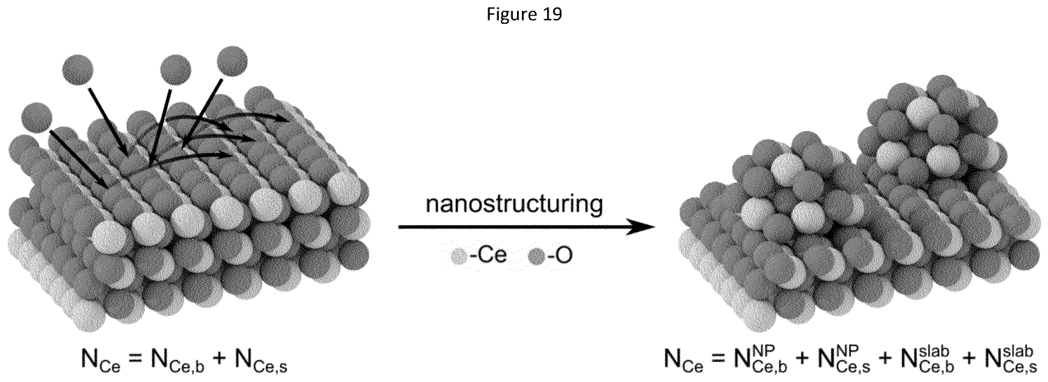

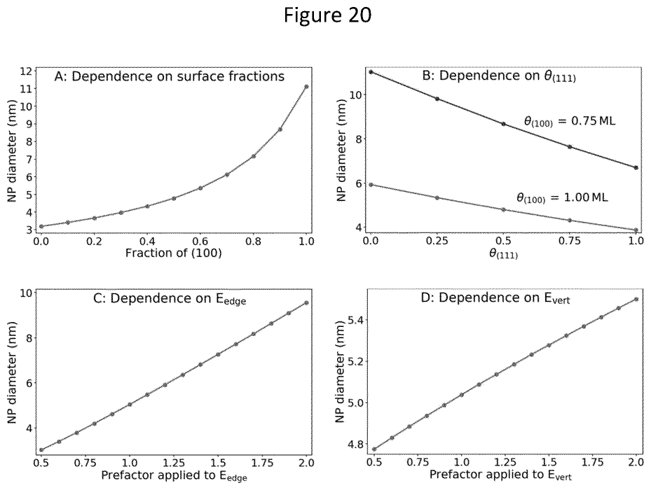

To understand the rationale behind the formation of ceria NPs observed by STM (see FIG. 7a-c) a thermodynamic model was considered under the assumption that the material forming the ceria NPs stems from the top CeO₂(111) layers of the sample (FIG. 19). Consequently, the ceria film became thinner, while the total number of ceria formula units remained constant during the restructuring (Ce atoms are not sputtered by the relatively light 0 atoms in the plasma). A ceria NP was approximated as a hemisphere that grows in registry with the underlying film and employed surface energies computed for different peroxo-covered extended surfaces (Tables 5-9), as well as step and corner energies, using calculations of high Miller-index surfaces (Tables 10⁻¹¹). Depending on the input parameters, the ceria NPs were between 3 and 11 nm in diameter (FIG. 20), i.e., larger than experimentally observed. Nonetheless, these thermodynamic considerations demonstrate that the oxygen plasma assists the surface restructuring and formation of ceria NPs on the initially flat film in addition to the possible bombardment effects of the high-energy 0 atoms in the plasma. The increase in surface area led to a larger number of local surface structures that can stabilize oxygen radicals via the peroxo groups, while the exposure of facets other than (111) on ceria NPs simultaneously entailed a more exothermic reaction energy for the formation of peroxides.

TABLE 5

| | A_{surf} | A_{surf}/Ce | γ | γ | Eᵣₑₗₐₓ |
|---|---|---|---|---|---|
| Surface | [Å²] | [Å²] | [J/m²] | [eV/Å²] | [eV] |
| (111) | 52.228 | 13.057 | 0.698 | 0.044 | −0.011 |
| (110) | 85.288 | 21.322 | 1.061 | 0.066 | −1.298 |
| (100) | 60.308 | 15.077 | 1.462 | 0.091 | −2.446 |

TABLE 6
Θ_{peroxide} [ML]

| Surface | 0.25 | 0.5-diag | 0.5-row | 0.75 | 1.0 |
|---|---|---|---|---|---|
| (111) | −2.675 | −5.223 | −5.331 | −7.731 | −10.093 |
| | (−2.675) | (−2.547) | (−2.656) | (−2.400) | (−2.362) |
| (110) | −2.872 | −5.680 | −5.720 | −8.348 | −11.001 |
| | (−2.872) | (−2.809) | (−2.849) | (−2.628) | (−2.653) |
| (100) | −3.362 | −6.732 | −6.559 | −9.691 | −12.669 |
| | (−3.362) | (−3.370) | (−3.197) | (−2.959) | (−2.978) |

TABLE 7

| | Θ_{peroxide} (ML) | | | | | |
|---|---|---|---|---|---|---|
| Surface | 0.0 | 0.25 | 0.5-diag | 0.5-row | 0.75 | 1.0 |
| (111) | 0.044 | −0.008 | −0.056 | −0.059 | −0.104 | −0.150 |
| | (0.698) | (−0.123) | (−0.904) | (−0.937) | (−1.674) | (−2.398) |
| (110) | 0.066 | 0.033 | 0.000 | −0.001 | −0.032 | −0.063 |
| | (1.061) | (0.522) | (−0.006) | (−0.013) | (−0.507) | (−1.005) |
| (100) | 0.091 | 0.035 | −0.020 | −0.018 | −0.069 | −0.119 |
| | (1.462) | (0.568) | (−0.327) | (−0.281) | (−1.113) | (−1.904) |

TABLE 8
Θ_{peroxide} [ML]

| Surface | 0.25 | 0.5-diag | 0.5-row | 0.75 | 1.0 |
|---|---|---|---|---|---|
| (111) | −2.175 | −4.221 | −4.330 | −6.228 | −8.090 |
| | (−2.175) | (−2.046) | (−2.155) | (−1.899) | (−1.862) |
| (110) | −2.371 | −4.679 | −4.719 | −6.846 | −8.997 |
| | (−2.371) | (−2.308) | (−2.348) | (−2.127) | (−2.152) |
| (100) | −2.861 | −5.731 | −5.558 | −8.189 | −10.666 |
| | (−2.861) | (−2.869) | (−2.696) | (−2.458) | (−2.477) |

TABLE 9

| | Θ_{peroxide} (ML) | | | | | |
|---|---|---|---|---|---|---|
| Surface | 0.0 | 0.25 | 0.5-diag | 0.5-row | 0.75 | 1.0 |
| (111) | 0.569 | 0.025 | −0.486 | −0.514 | −0.988 | −1.454 |
| (110) | 1.413 | 0.820 | 0.243 | 0.233 | −0.299 | −0.837 |
| (100) | 1.375 | 0.660 | −0.057 | −0.014 | −0.672 | −1.291 |

TABLE 10

| Input | Value | | Description |
|---|---|---|---|
| γ | -0.039 | eV/Å² | Surface energy of (111)-0.5 ML |
| γ̅ | 0.069 | eV/Å² | 0.5 γ^{(111)-0.5ML} + 0.5 γ^{(100)-1.0ML} |
| | | | as effective surface energy for ceria NP |
| R | 2.039 | Å | Radius of a Ce atom calculated |
| | | | from its exposed area on (111) |
| E_{edge} | 0.303 | eV/Å | Edge energy contribution per Å |
| Eᵥₑᵣₜ | 1.817 | eV/Ce | Energy contribution per vertex |
| | | | Ce atom on the CeOₓ NP |

TABLE 11

| | A_{surf} | Ce_{surf} | Lₛₜₑₚ | γ | γ | Eₛₜₑₚ |
|---|---|---|---|---|---|---|
| Surface | [Å²] | (Ceₛₜₑₚ) | [Å] | [J/m²] | [eV/Å²] | [eV] |
| (112) | 36.931 | 3 (1) | 4.246 | 0.987 | 0.062 | 1.137 |
| (221) | 45.231 | 3 (1) | 3.285 | 0.834 | 0.052 | 1.217 |
| (311) | 50.004 | 4 (2) | 4.194 | 1.133 | 0.071 | 1.199 |

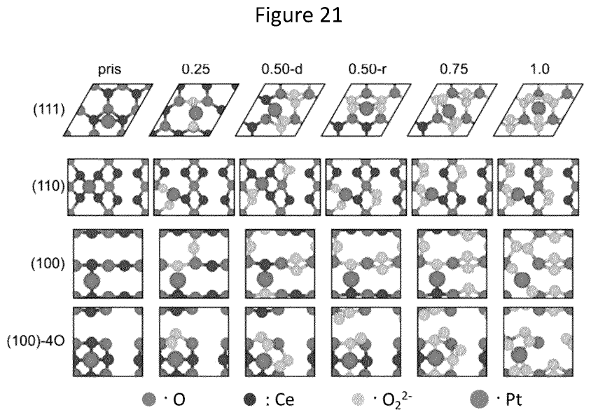

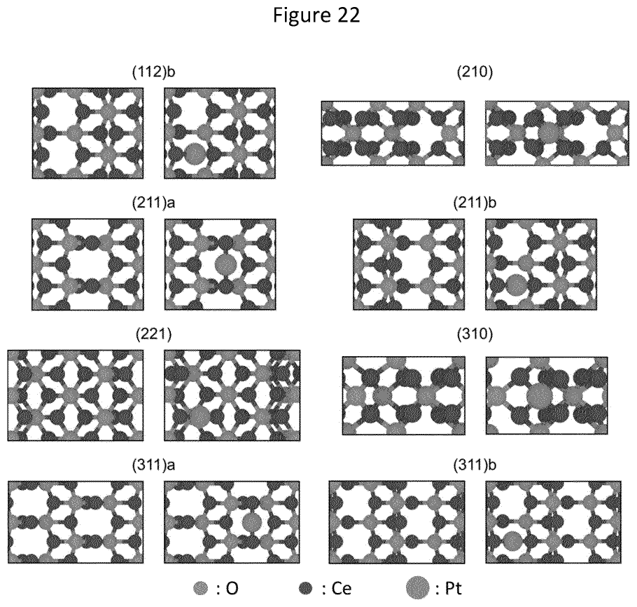

Not surprisingly, peroxo species affected the Pt adsorption on the CeO₂ surfaces (Table 12 and FIG. 21). As compared to pristine CeO₂(111), a Pt atom was bound to the peroxo-covered (111) surface by 1-2 eV more strongly, depending on the peroxide coverage. The effect was smaller for the (110) surface (the energy gain is about 0.6 eV, on average), while on the (100) surface, peroxo species may even weaken the Pt bonding. Among the surfaces studied, the strongest Pt adsorption was observed for (100)-40 coordination environment. Interestingly, the presence of peroxo groups showed no beneficial effect in terms of Pt bonding for this site. Pt adsorption on stepped surfaces was also examined (FIG. 22). Even though square-planar 40-coordination environments, particularly on the (210) and (310) surfaces, can again provide sites for strongly exothermic Pt adsorption (Table 12), these stepped surfaces exhibited considerably higher surface energies and were therefore thermodynamically unstable. Table 13 shows adsorption energies (in eV) of a Pt single atom adsorbed in the most stable structure on clean and peroxide-covered CeO₂ surfaces as a function of the peroxide coverage (1 ML corresponds to one peroxo group per (1×1) surface unit cell). At 0.5 ML coverage for the (2×2) slabs used in the calculations, two peroxo groups allow for two non-equivalent geometric arrangements: 1) along the unit vectors (labelled “r” =row-wise); and 2) diagonal to the unit vectors (“d”=diagonal). The energies are referenced to the corresponding ceria surfaces and Pt bulk. Negative values indicate exothermic adsorption and unfavorable Pt aggregation (Pt—Pt bond formation).

TABLE 12

| | A_{surf} | γ | γ | ΔE_{ads}^{Pt} | | ΔE_{ads}^{O} |
|---|---|---|---|---|---|---|
| Surface | [Å²] | [J/m²] | [eV/Å²] | [eV] | Pt-nO | [eV] |
| (112)b | 36.931 | 0.987 | 0.062 | 0.590 | 2O | −3.278 |
| (210) | 67.426 | 1.945 | 0.121 | −3.581 | 4O | −5.321 |
| (211)a | 36.931 | 1.252 | 0.078 | 0.172 | 2O | −4.181 |
| (211)b | 36.931 | 1.315 | 0.082 | 0.054 | 2O | −3.281 |
| (221) | 45.231 | 0.834 | 0.052 | 1.425 | 2O | −2.763 |
| (310) | 47.677 | 1.807 | 0.113 | −0.734 | 4O | −4.699 |
| (311)a | 50.004 | 1.346 | 0.084 | 1.111 | 2O | −2.784 |
| (311)b | 50.004 | 1.133 | 0.071 | 0.626 | 2O | −3.269 |

TABLE 13

Θ_{peroxide} (ML)

| Surface | 0.0 | 0.25 | 0.5-d | 0.5-r | 0.75 | 1.0 |
|---|---|---|---|---|---|---|
| (111) | 2.80 | 1.28 | 1.98 | 1.75 | 1.62 | 0.59 |
| (110) | 0.60 | −0.17 | 0.75 | −0.08 | 0.11 | 0.12 |
| (100) | 0.31 | 0.14 | 0.46 | 0.30 | 0.56 | 0.64 |
| (100)-4O | −0.85 | −0.59 | −0.95 | −0.24 | −0.63 | 1.76 |

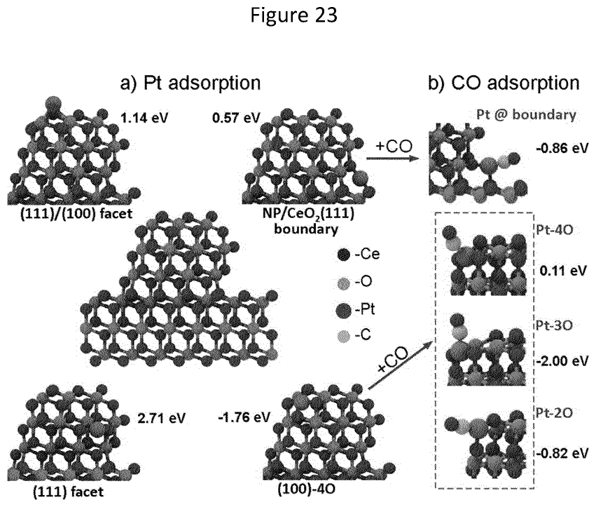

Next, the interaction of Pt single atoms with ceria NPs formed on the CeO₂(111) surface, as shown by STM, was investigated. To this end, a model system was constructed consisting of a ceria cluster anchored on an extended CeO₂(111)-(7×7) slab. The particle size was adapted to that measured experimentally (i.e., about 1.0 nm, see FIG. 7), and the entire structure was optimized by DFT accordingly (FIG. 23a, center). Obviously, new Pt adsorption sites were formed at the boundary between the NP and the surrounding (111) terrace. The computed adsorption energy of a Pt single atom (E_{ads}(Pt)) on these sites (0.57 eV) was substantially smaller than on clean and peroxo-covered (111) surfaces (2.80 and 1.3-1.6 eV, respectively, Table 13). However, the (100)-40 sites exposed at the vertices of the ceria NPs presented the thermodynamically most favorable adsorption sites for Pt (E_{ads}(Pt)=−1.76 eV).

Depending on their location on the ceria NP the coordination environments for adsorbed Pt atoms were chemically different from those of their symmetrically equivalent counterparts on the extended ceria surfaces. In particular, at (100)-40 sites, the difference amounted to 0.9 eV (i.e., —1.76 vs −0.85 eV, on NP vertices and the corresponding extended surface, respectively).

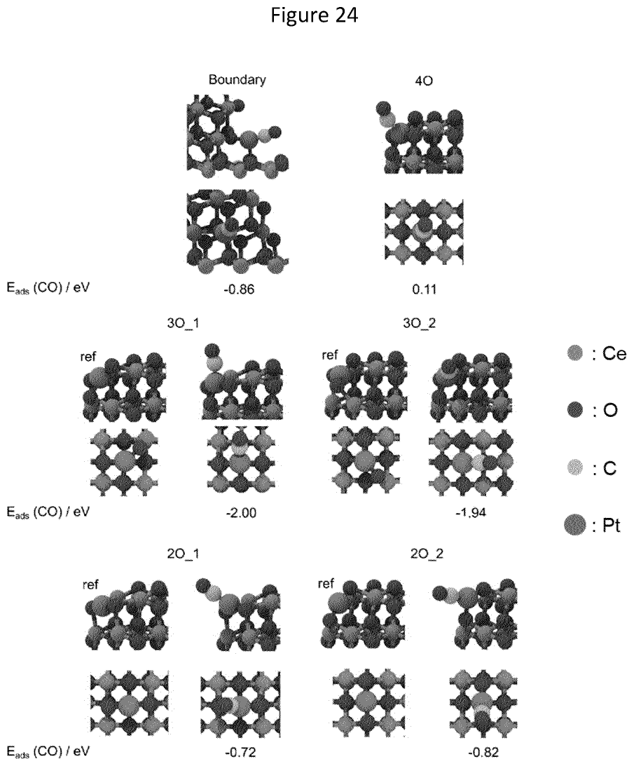

The NP/terrace boundary and the square-planar “nanopocket” Pt-binding sites of this system for CO adsorption were also investigated (FIG. 23b, FIG. 24). CO did not adsorb on Pt in the (100)-40 site (E_{ads}(CO)=0.11 eV), while CO adsorption was exothermic for Pt at the NP/CeO₂(111) boundary (E_{ads}(CO)=−0.86 eV). However, on this site the CO molecule was oriented almost parallel to the metal support surface and, therefore, will be invisible to IRAS due the above-mentioned metal selection rules. Both these findings may explain the very low intensity of the IRAS bands on the “as deposited” Pt/CeO₂-plasma surface as compared to that observed for the Pt/CeO₂(111) system (FIG. 10). As for the latter, the calculations performed for a Pt ad-atom on the pristine CeO₂(111) surface yielded E_{ads}(CO)=−2.14 eV and a CO frequency of 2093 cm⁻¹, which matches the 2087 cm⁻¹ band observed on the “as deposited” Pt/CeO₂(111) sample (FIG. 10). The CO binding energy increased to −2.33 and −2.62 eV for Pt adsorbed onto 0.25 and 0.5 ML peroxo-covered CeO₂(111) surfaces, and the CO frequency shifted to 2081 and 2080 cm⁻¹, respectively. Nonetheless, the IRAS band at 2104 cm⁻¹ observed in the Pt/CeO₂ sample can be assigned to Pt single ad-atoms adsorbed on the bare and/or peroxo-covered CeO₂(111) surface. Upon UHV annealing at 500 K, these atoms probably migrate to the more strongly bound boundary and nanopocket sites where they only weakly adsorb CO that leads to considerable reduction of the signal intensity as shown in FIG. 10b.

In an attempt to identify the nature of strongly bonded CO ad-species remained on the Pt/CeO₂-plasma surface after reaction, several structures were computed derived from the Pt/nanopocket site by sequential removal of 0 atoms from the Pt-40 coordination as a result of their reaction with CO. The resulting structures, denoted Pt-30 and Pt-20 in FIG. 23b, revealed exothermic CO adsorption with energies amounting to −2.00 eV and −0.82 eV, respectively. As such, the Pt-30 structure appears as likely candidate responsible for CO observed by IRAS on the post-reacted Pt/CeO₂-plasma sample.

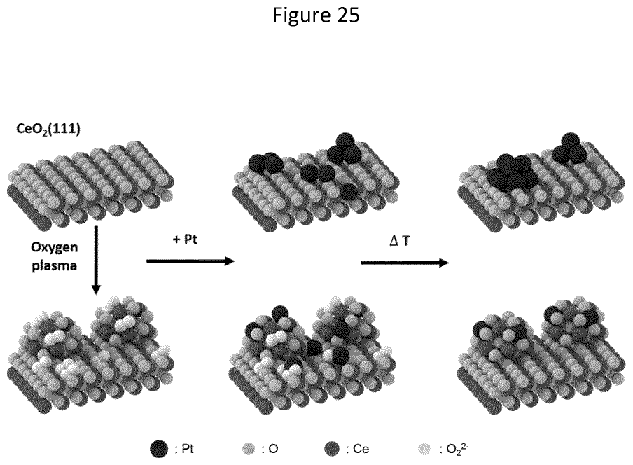

Based on the above observations, the overall impact of the plasma treatment on the structure of the Pt/CeO₂(111) surface is rationalized as shown in FIG. 25. It is postulated that the plasma treatment leads to two modifications of the ceria surface: 1) morphological, as evident from the pronounced nanostructuring resulting in the formation of ceria nanoparticles; and 2) chemical, via surface peroxide formation. During Pt deposition, the metal atoms may either directly stick to the ceria NPs or adsorb onto the peroxo-covered CeO₂(111) surface in-between, where they form isolated strongly bound complexes with the surface peroxide groups, in contrast to the pristine CeO₂(111) film surface, where Pt atoms diffuse on the surface and agglomerate into Pt NPs. With increasing temperature, the peroxo-trapped Pt atoms on the (111) terraces may further migrate to the numerous ceria NPs around and occupy sites at the particle/terrace boundary and the nanopockets. Thus, the peroxide-covered CeO₂(111) surface may, firstly, prevent Pt aggregation during deposition and, secondly, act as a reservoir for Pt single atoms on ceria NPs. Consequently, the morphological and chemical modifications of the ceria surface, as well as their synergistic interplay may be beneficial to achieve a high density of stable Pt single atoms.

1. A catalytically active material comprising
a support comprising a metal oxide, and
atomically dispersed noble metal on the surface of the support, wherein
the metal oxide is selected from TiO₂, CeO₂, ZnO, SnO₂, Ga₂O₃, In₂O₃, ZrO₂, and Fe₂O₃,
the noble metal is selected from Pt, Pd, Rh, and Au, and
the catalytically active material is obtainable by a method comprising a step of non-thermal plasma treatment in the presence of O₂.

2. The catalytically active material according to claim 1, wherein
the number of the noble metal atoms normalized to the surface area of the catalytically active material is from 3.0×10¹² at/cm² to 8.0×10¹⁴ at/cm².

3. The catalytically active material according to claim 1, wherein
the relative intensity of the noble metal in oxidation state X with respect to the total intensity of the noble metal of all oxidation states in the catalytically active material is 50% or more, as measured by X-ray photoelectron spectroscopy, wherein
X is 2+ for Pt and Pd, and X is 3+ for Rh and Au.

4. The catalytically active material according to claim 1, wherein
an infrared absorption spectrum measured on the catalytically active material shows an absorption band within the range of 2085 cm⁻¹ to 2120 cm⁻¹ that does not change upon heating the material from 300 K to 500 K, when measured under the following condition:
(1) exposing the catalytically active material in a reaction cell to a reaction mixture consisting of 1% CO and 5% O₂ balanced by Ar at 1 bar,
(2) heating the catalytically active material to 500 K at a rate of 60 K/min and reacting the mixture for 5 min at 1 bar at 500 K,
(3) cooling down the reaction mixture and the catalytically active material to 300 K,
(4) removing the reaction mixture from the reaction cell and applying ultrahigh vacuum conditions of 10⁻⁹ mbar or less,
(5) recording a spectrum in ultrahigh vacuum at 300 K without additional exposure to CO,
(6) increasing the temperature to 500 K at a rate of 60 K/min and recording spectra at 320 K, 360 K, 400 K, 440 K, 480 K and 500 K, and
(7) comparing the absorption maxima in the spectra recorded at the respective temperatures.

5. The catalytically active material according to claim 1, wherein the metal oxide and the noble metal are selected from the following material combinations:
the metal oxide is selected from TiO₂ and CeO₂ and
the noble metal from Pt and Pd, wherein
the metal oxide is CeO₂ and the noble metal is Pt.

6. The catalytically active material according to claim 1, wherein
the support is in the form of particles or powder.

7. The catalytically active material according to claim 6, wherein the support consists of the metal oxide.

8. The catalytically active material according to claim 6, wherein
the powder support has an average particle diameter of 10 nm to 50 nm, as measured by scanning transmission electron microscopy, and is CeO₂ powder support.

9. (canceled)

10. The catalytically active material according to claim 1, wherein
the support is in the form of a film.

11. The catalytically active material according to claim 10, wherein
the support consists of a substrate and clusters supported on the substrate,
the substrate and the clusters comprise the same metal oxide, and
the average diameter of the clusters is 2.0 nm or less as determined by scanning tunneling microscopy.

12. The catalytically active material according to claim 11, wherein the substrate and the clusters consist of the same metal oxide.

13. A method for a catalytic oxidation of CO to CO₂, comprising using the catalytically active material according to claim 1, wherein the catalytically active material is used as follows:
a gas mixture comprising CO and O₂ is reacted in the presence of the catalytically active material at a pressure of 0.8 to 2.0 bar, and at reaction temperatures of 323 K to 573 K, wherein
the content of CO in the gas mixture is 5 vol % or less,
the content of O₂ in the gas mixture is 2 vol % or more and 25 vol % or less, and
the CO/O₂ molar ratio is 2.0 or less.

14. A method for a catalytic hydrogenation of alkyne,
comprising using the catalytically active material according to claim 1, wherein the catalytically active material is used as follows:
a gas mixture comprising an alkyne and H₂ is reacted in the presence of the catalytically active material at a pressure of 0.8 to 2.0 bar, at a temperature of 323 K to 573 K, wherein
the content of the alkyne in the gas mixture is 10 vol % or less,
the alkyne to H₂ molar ratio in the gas mixture is 1 or less, and
the alkyne is a C₂-C₅ alkyne, and more C₂-C₃ alkyne.

15. A method for producing the catalytically active material according to claim 6, comprising the steps of
(1) providing a precursor for the catalytically active material comprising a support in the form of particles comprising a metal oxide and noble metal on the surface of the support,
(2) calcining the precursor for the catalytically active material in the presence of O₂, and
(3) carrying out a non-thermal plasma treatment of the calcined precursor for the catalytically active material in the presence of O₂.

16. The method according to claim 15, wherein the step (1) is selected from the following steps (1a) and (1b):
(1a) depositing the noble metal onto the support from an aqueous solution of a salt of the noble metal, wherein the support consists of the metal oxide, and
(1b) co-precipitating the noble metal and the metal oxide from an aqueous solution containing salts of the noble metal and the metal oxide.

17. The method according to claim 15, wherein the non-thermal plasma treatment is carried out at a pressure of 20 mbar or less and at an O₂ content of 5 vol % or more.

18. A method for producing the catalytically active material according to claim 10 selected from Methods A and B,
Method A comprising steps of:
(1) providing a metal oxide film support,
(2) carrying out a non-thermal plasma treatment in the presence of O₂ to obtain a plasma-treated metal oxide film support, and
(3) depositing noble metal onto the plasma-treated metal oxide film support in gas phase by physical vapor deposition of noble metal atoms;
Method B comprising steps of:
(1) providing a metal oxide film support,
(2) depositing noble metal onto the metal oxide film support from a solution containing the noble metal, and
(3) carrying out a non-thermal plasma treatment in the presence of O₂;
wherein the non-thermal plasma treatment is carried out at a pressure of 1×10⁻⁶ mbar to 1×10⁻⁵ mbar of O₂.

19. The catalytically active material according to claim 2, wherein
the number of the noble metal atoms normalized to the surface area of the catalytically active material is from 3.0×10¹² at/cm² to 4.0×10¹⁴ at/cm² or from 6.0×10¹² at/cm² to 2.0×10¹⁴ at/cm².

20. The catalytically active material according to claim 3, wherein
the relative intensity of the noble metal in oxidation state X with respect to the total intensity of the noble metal of all oxidation states in the catalytically active material is 75% or more, as measured by X-ray photoelectron spectroscopy, wherein
X is 2+ for Pt and Pd, and X is 3+ for Rh and Au.

21. The catalytically active material according to claim 11, wherein
the average diameter of the clusters is 1.5 nm or less as determined by scanning tunneling microscopy.
